(19) United States
(12) Patent Application Publication
(10) Pub. No.: US 2004/0146858 A1
(43) Pub. Date: Jul. 29, 2004

(54) Novel siRNA gene libraries and methods for their production and use

(75) Inventors: Henry Li, Carlsbad, CA (US); Jon E. Chatterton, San Diego, CA (US); Ning Ke, San Diego, CA (US); Flossie Wong-Staal, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Immusol, Inc., San Diego, CA

(21) Appl. No.: 10/626,512

(22) Filed: Jul. 23, 2003

Related U.S. Application Data
(60) Provisional application No. 60/398,915, filed on Jul. 24, 2002.

(51) Int. Cl. C12Q 1/70; C07H 21/04; C12N 7/00; C12N 15/86

(52) U.S. Cl. 435/5; 435/456; 435/235.1; 536/23.72

(57) ABSTRACT

The present invention relates to methods and compositions for the elucidation of gene function and the identification of novel genes. Specifically, the present invention relates to methods and compositions for improved functional genomic screening, functional inactivation of specific essential or non-essential genes, and identification of genes that are modulated in response to specific stimuli or encode recognizable phenotypic traits. In particular, the compositions of the present invention include, but are not limited to, expression cassettes comprising a novel dual promoter transcription system, that utilizes modified promoters, preferably containing complementary termination sequences, positioned across a coding sequence and in opposite orientation to each other. In addition, the present invention includes libraries comprising the expression cassettes of the invention, including vectors for transforming cells, such as replication-deficient retroviral vectors. The invention also includes methods for the production and screening of dsRNA/siRNA libraries, as well as therapeutic uses for the siRNAs expressed in accordance with the invention.

### CROSS-REFERENCE TO RELATED APPLICATIONS

This application claims priority of Provisional Application Serial No. 60/398,915 which was filed with the U.S. Patent and Trademark Office on Jul. 24, 2002.

### FIELD OF THE INVENTION

Generally, the present invention relates to the field of functional genomics. Specifically, the invention relates to a novel method for generating randomized siRNA gene libraries and the use of such libraries for the discovery of cellular genes associated with disease processes.

### BACKGROUND OF THE INVENTION

The human genome project and allied interests will soon have elucidated the sequence of the entire human genome [Cox et al., Science, 265:2031-2031 (1994); Guyer et al., Proc. Natl. Acad. Sci. USA, 92:10841-10848 (1995)]. While this anticipated advance is exciting, it is also misleading since knowledge of the sequences of open reading frames and genetic coding regions, without a knowledge of the function of the gene products of this vast array of putative genes, provides only very limited insight into the human genome. Full knowledge of the genome requires knowledge of the function of each of the gene products of the putative genetic coding sequences. While gene function determination is ongoing within the field of molecular genetics, the rate at which the function of a gene can be determined is many orders of magnitude slower than the rate at which a gene can be sequenced. Therefore, a massive backlog of genetic sequences in search of a function looms on the horizon.

Small interfering RNAs (siRNA) are short double-stranded RNA fragments that elicit a process known as RNA interference (RNAi), a form of sequence-specific gene silencing. Zamore, Phillip et al., Cell, 101:25-33 (2000); Elbashir, Sayda M., et al., Nature 411:494-497 (2001). siRNAs are assembled into a multicomponent complex known as the RNA-induced silencing complex (RISC). The siRNAs guide RISC to homologous mRNAs, targeting them for destruction. Hammond et al., Nature Genetics Reviews 2:110-119 (2000). RNAi has been observed in a variety of organisms including plants, insects and mammals, and cultured cells derived from these organisms. The development of efficient methods for screening effective siRNAs offers a means for identifying the functional characteristics of genes silenced by such siRNAs, through a process of subtractive phenotypic analysis, a technology developed by the Assignee hereof known as Inverse Genomics®. Discovery of efficient screening techniques would also provide a method for screening prospective therapeutic compounds comprising siRNA molecules, thus advancing the field of gene therapy. For a review of RNAi and siRNA expression, see Hammond, Scott M et al., Nature Genetics Reviews, 2:110-119; Fire, Andrew, TIG, 15(9):358-363 (1999); Bass, Brenda L., Cell, 101:235-238 (2000).

### SUMMARY OF THE INVENTION

The present invention provides DNA expression cassettes, transgenic retrovirus constructs and libraries of the same for the production and expression of dsRNA molecules of known and random sequences, in particular, siRNAs. The invention also provides methods for the construction and use of the DNA expression cassettes, transgenic retrovirus constructs and libraries of the invention.

In one embodiment, the invention provides a DNA expression cassette comprising a double-stranded DNA sequence between 16-25 bases long, more preferably between 17-23 bases long, and most preferably between 18-21 bases long. This DNA may comprise either a known or randomized nucleotide sequence. The double-stranded DNA sequence has a first and a second end, with each end operably linked to a pol III promoter. Each pol III promoter has a TATA box, and is modified by substitution. One substitution places at least four consecutive adenylyl residues 3′ to the TATA box. A second optional substitution of between 1 to 20 bases can be made 5′ to the at least four consecutive adenylyl residue substitution and 3′ to the TATA box. Constructing the expression cassette in this manner results in production of a dsRNA with a 3′ overhang of two or more nucleotides when the double stranded DNA sequence is transcribed from both pol III promoters.

The two promoters of the invention may be the same promoters or they may be different. For example, the promoters may both be H1 RNA promoters or U6 snRNA promoters, or one promoter may be a H1 RNA promoter and the other promoter may be a U6 snRNA promoter. Alternatively, one promoter may be a human U6 snRNA promoter and the other may be a murine U6 snRNA promoter. In another embodiment, one or both of the promoters may be a tRNA promoter (e.g., the tRNA^{Val} promoter).

The promoters of the present invention may also be made inducible by incorporating an inducible operator sequence 5′ to the TATA box. When the operator is induced, a dsRNA is transcribed from the pol III promoters. In some aspects of the present invention, this inducible operator sequence is the tet-o operator.

In a preferred aspect of the present invention, the DNA sequence is randomized. Other aspects initiate transcription of each strand at a G or an A, followed by transcription of the remainder of the DNA sequence. The DNA expression cassette of the present invention may also be part of a nucleic acid packaged into a viral particle or may be part of a self-replicating DNA.

The invention also includes libraries of DNA expression cassettes of the present invention. The promoters of such expression cassette libraries may be inducible by inclusion of an appropriate operator sequence into the promoters, as noted above. In certain embodiments, each DNA expression cassette of such libraries is packaged in a viral particle. In other embodiments, each DNA expression cassette is included in a cell genome, or in a self-replicating construct.

The invention also includes a recombinant retrovirus comprising a genome which, when converted to the proviral form through the action of reverse transcriptase, includes a double-stranded DNA sequence between 16-25 bases long, more preferably between 17-23 bases long, most preferably between 18-21 bases long. This double-stranded DNA can have either a known or randomized nucleotide sequence. The double-stranded DNA sequence has a first and a second end, with each end operably linked to a pol III promoter. Each pol III promoter has a TATA box, and is modified by substitution. One substitution places at least four consecutive adenylyl residues 3′ to the TATA box. A second optional substitution of between 1 to 20 bases can be made 5′ to the at least four consecutive adenylyl residue substitution and 3′ to the TATA box. Constructing the expression cassette in this manner results in production of a dsRNA with a 3′ overhang of two or more nucleotides when the double stranded DNA sequence is transcribed from both pol III promoters.

The invention includes methods for producing and using the expression cassettes of the present invention. One such method comprises synthesizing a single-stranded coding sequence, constructing a vector comprising the two opposing promoters, inserting the coding sequence between two promoters of the vector, and generating a complementary strand to the single-stranded coding sequence, thereby forming an expression cassette of the present invention. As noted above, these expression cassettes optionally may be inducible and may contain optional substitutions within their promoter regions.

In one aspect of this method, the complementary strand to the single stranded DNA sequence is generated by competent bacteria after transformation with the expression vector comprising the single stranded DNA sequence. Alternatively, the complementary strand may be synthesized in vitro using Klenow polymerase and a DNA ligase.

In certain aspects of the method, the optional 1 to 20 base substitution within the promoter comprises at least one restriction site. In another aspect, a guanylyl residue is inserted at the 5′ end of the single-stranded DNA sequence, and a cytosyl residue at the 3′ end. Alternatively, an adenylyl residue may be inserted at the 5′ end of the single-stranded DNA sequence, in which case a thymidyl residue is inserted at the 3′ end. These additional bases are included in some constructs to meet the recognition requirements of some polymerases.

Another embodiment of the invention is a method for producing a library of DNA expression cassettes of the invention. This method comprises first synthesizing a plurality of single-stranded DNA sequences between 16 and 25 bases long, preferably between 17-23 bases long, most preferably between 18-21 bases long. The DNA sequences may be either known or randomized. Once these DNA sequences have been synthesized, the expression cassettes can be constructed as noted above, with each cassette containing one of the plurality of the single-stranded sequences.

In addition to methods of construction, the invention also includes methods of use. For example, one method of the invention correlates expression of an siRNA transcription sequence with a phenotypic change in a cell resulting from silencing of a cellular gene by the siRNA, where expression of the cellular gene has not been previously characterized as contributing to the phenotypic change. This method comprises introducing to a cell population (e.g., by viral transduction) a library of exogenous randomized siRNA genes each of which is incorporated within an expression cassette in accordance with the present invention. The transformed cell population is then screened to detect a phenotypic difference between the cells of the population introduced to the library of siRNA genes and those cells not introduced to the library. Once a phenotypic difference is detected, the siRNA gene of the library responsible for the phenotypic change is identified. In some aspects of the method, the identified siRNA gene of the library responsible for the phenotypic change is isolated.

In some aspects of this method, the step of detecting a phenotypic difference comprises observation of a difference in cellular growth between the cells of the population introduced to the library of dsRNA genes and those cells not introduced to the library. In other aspects, the detecting step comprises co-expression of a detectable marker by the cells of the population introduced to the library of dsRNA genes. In certain aspects, the detectable marker may be a fluorescent protein or a cell surface protein, or antibiotic resistance.

The cell population may be a eukaryotic cell population. The siRNA identified and/or isolated may be an siRNA that inhibits cell division. Other identified and/or isolated siRNAs may inhibit viral gene expression. Others may inhibit cell death initiated by inducers of apoptosis/necrosis. Still others may inhibit excretion of an extracellular protein, expression of a cell surface marker, or a genetic suppressor.

Another embodiment of the invention is a method of regulating the transcription of siRNA in a cell. The method involves introducing into the cell a vector that includes an expression cassette of the invention that comprises a promoter that is inducibly regulated. In these systems, transcription of the double-stranded DNA to produce an siRNA is initiated by inducing the inducible operator. The effect on the cell of transcription of the siRNA produced using this inducible operator method may then be determined, based on any of a number of factors, including the inhibition of cell division, cell death, viral gene expression, excretion of an extracellular protein, expression of a cell surface marker, a genetic suppressor or a signal transduction pathway.

Yet another embodiment of the invention is a method of transducing a cell. The method comprises obtaining a transgenic retrovirus having a genome including an expression cassette of the present invention. The cell is then transduced with the transgenic retrovirus. Whether transduction has occurred is determined by observation of the presence or absence of a detectable cellular trait associated with the siRNA, e.g., inhibition of cell division, cell death, viral gene expression, excretion of an extracellular protein, cell surface marker, a genetic suppressor or a signal transduction pathway. Alternatively, whether transduction has occurred may be determined by incorporating into the viral genome an expression cassette for an appropriate detectable marker, e.g., a fluorescent protein, cell surface marker, or antibiotic resistance.

### BRIEF DESCRIPTION OF THE DRAWINGS

FIG. 1 is a schematic depiction of an exemplary DNA expression cassette constructed in accordance with the present invention including two opposing U6 promoters.

FIG. 2 is a schematic depiction of the construction of an exemplary DNA expression cassette in accordance with the present invention in which the DNA sequence is randomized and in which the cassette is incorporated into a retroviral vector, pLPR.

FIG. 3 depicts a human U6 snRNA promoter, modified to contain the Tet-o operator between the PSE and TATA box elements of the promoter.

FIG. 4 depicts a U6 snRNA promoter with four adenylyl residues at the extreme 3′ end of the promoter which are complementary to the termination sequence for a polymerase transcribing the opposing strand. In the region 5′ to this sequence of four adenylyl residues and 3′ to the TATA box, up to 20 bases which may be substituted to incorporate nucleic acid sequences for restriction sites, operator elements or other sequence desirable for facilitating cloning or controlling expression.

FIG. 5 is a western blot showing p53 protein expression in MCF-7 cells after transduction with a retroviral vector carrying a dual promoter expression cassette in accordance with the invention engineered to express p53 siRNA.

FIG. 6A is a western blot showing p53 protein expression in A431 cells after transduction with (i) a lentiviral vector carrying a single murine U6 promoter hairpin siRNA expression cassette engineered to express p53 siRNA; and (ii) a lentiviral vector carrying a dual promoter expression cassette in accordance with the invention engineered to express p53 siRNA.

FIG. 6B is a western blot showing p53 protein expression in A431 cells after transduction with (i) a retroviral vector carrying a single murine U6 promoter hairpin siRNA expression cassette engineered to express p53 siRNA; and (ii) a retroviral vector carrying a dual promoter expression cassette in accordance with the invention engineered to express p53 siRNA.

### DEFINITIONS

The term “cellular gene” or “gene” refers to a nucleic acid fragment that encodes a specific transcription product and includes regulatory sequences preceding (5′ non-coding) and following (3′ non-coding) the coding region that control transcriptional expression.

The term “cell division” refers to the physical cellular event, and preceding biochemical events, that culminate in a cell splitting into two autonomous units.

The term “cellular growth” refers to those cellular processes that lead to an increase in cell mass, volume or number.

The term “cell population” generally refers to a grouping of cells of a common type, typically having a common progenitor, although the phrase is also applicable to heterogeneous cell populations.

The term “cell surface protein” refers to any biological molecule at least a portion of which is associated with the outer surface of a cell membrane and which comprises proteinaceous material.

The term “competent bacteria” refers to prokaryotic cells capable of being transformed with exogenous nucleic acid, or transduced using a viral system.

The terms “detectable marker”, “detectable trait” and “detectable cellular trait” refer to any physical or chemical characteristic expressed by a cell that can be identified by observation or test.

The term “phenotypic change” refers to any change in physical, morphologic, biochemical or behavioral characteristics of a cell that can be identified by observation or test.

The term “exogenous” refers to any molecule or agent that is foreign to its current environment, as in originating, being derived or developing from a source other than the current environment.

The term “extracellular protein” refers to any material, at least partially proteinaceous in character, located outside of a cell.

The term “fluorescent protein” refers to any material, at least partially proteinaceous in character, capable of emitting fluorescent energy in response to excitement resulting from exposure to electromagnetic waves (e.g. UV, etc.).

The term “gene expression” refers to all processes involved in producing a biologically active agent, whether nucleic acid or protein, from a nucleic acid encoding the biologically active agent. Gene expression includes all post-transcriptional and/or post-translational processing required to produce the mature agent.

The term “genetic suppressor” refers to genetically active agents that inhibit or prevent gene expression.

“Inducible” means that a promoter sequence, and hence the nucleic acid sequence whose expression it controls, is subject to regulation in response to factors which act as “inducers”. These factors can be proteins, nucleic acids, small molecules or physical stimuli e.g. UV irradiation. Induction of regulated nucleic acid sequences may involve the binding of factors that directly stimulate activity, or alternatively may require the removal of factors so as to derepress expression of a nucleic acid sequence. Induction can be measured, for example by treating cells with a potential inducer and comparing the expression of a nucleic acid sequence in the induced cells to the activity of the same nucleic acid sequence in control samples not treated with the inducer. Control samples (untreated with inducers) are assigned a relative activity value of 100%. Induction of a nucleic acid sequence is achieved when the activity value relative to the control (untreated with inducers) is 110%, more preferably 150%, more preferably 200-500% (i.e., two to five fold higher relative to the control), more preferably 1000-3000% higher.

The term “Klenow polymerase” is the polymerase activity remaining after treatment of DNA polymerase I with the protease subtilisin to eliminate the 5′→3′ exonuclease activity of the holoenzyme.

The term “opposing nucleic acid strand” refers to a nucleic acid strand complementary and lying parallel to a reference strand. “Opposing nucleic acid strand”, unless otherwise stated, also infers that the opposing nucleic acid strand and the reference strand are annealed in a duplex predominantly through Watson-Crick base pairing.

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof.

“dsRNA” refers to an RNA molecule comprising two complementary RNA strands hybridized together through base pairing interactions. “siRNA” refers to a dsRNA that is preferably between 16 and 25, more preferably 17 and 23 and most preferably between 18 and 20 base pairs long, each strand of which has a 3′ overhang of 2 or more nucleotides. Functionally, the characteristic distinguishing an siRNA over other forms of dsRNA is that an siRNA is capable of specifically inhibiting expression of a gene by a process termed “RNA interference”.

A “library” refers to a collection of nucleic acid sequences that is representative of a defined biological unit. For example, a library of nucleic acids can be representative of all possible configurations of a nucleic acid sequence over a defined length. Alternatively, a nucleic acid library may be a collection of sequences that represents a particular subset of the possible sequence configurations of a nucleic acid of a defined length. A library may also represent all or part of the genetic information of a particular organism. Typically, a nucleic acid “library” is cloned into a vector, but this is not required.

A nucleic acid “library” of the present invention may be fully randomized, with the members of the collection showing no sequence preferences or constants at any position. Alternatively, the nucleic acid library may be biased. That is, some positions within the sequence are either held constant, or are selected from a limited number of possibilities. For example, in a preferred embodiment, the nucleotides are randomized with a bias favoring the proportions of bases in a given organism. The source of the randomized nucleic acid mixture can be from naturally-occurring nucleic acids or fragments thereof, chemically synthesized nucleic acids, enzymatically synthesized nucleic acids, or nucleic acids made by a combination of the foregoing techniques.

The term “signal transduction pathway” refers to those biochemical events whereby a chemical or physical event impinging upon a cell is transmitted to a cellular process leading to a change in the physical or metabolic state of the cell in response to the original chemical or physical event.

A “TATA box”, or “TATA element” refers to a nucleotide sequence element, common in many promoters, which binds a general transcription factor and hence specifies the position where transcription is initiated. The TATA box is an important element for transcription of sequences whose expression is dependent on type III RNA polymerase III promoters. In DNA constructs, as the name implies, the TATA box typically comprises the nucleic acid sequence 5′-TATA-3′, or variations thereof as known in the art.

A “promoter” refers to an array of nucleic acid control sequences that direct transcription of a nucleic acid. As used herein, a promoter includes necessary nucleic acid sequences near the start site of transcription, such as, in the case of a type III RNA polymerase III promoter, a TATA element. A promoter also optionally includes proximal and distal sequence elements, which can be located as much as several hundred base pairs from the start site of transcription. A “constitutive” promoter is a promoter that is active under most environmental and developmental conditions. An “inducible” promoter is a promoter that is active under environmental or developmental regulation. Thus, the term “promoter” means a nucleotide sequence that, when operably linked to a DNA sequence of interest, promotes transcription of that DNA sequence.

The term “promoter region” refers to a nucleotide region comprising a DNA regulatory sequence, wherein the regulatory sequence is derived from a gene which is capable of binding an RNA polymerase and initiating transcription of a given nucleic acid sequence. The “promoter region” of a given gene or set of genes, determines which of the three eukaryotic RNA polymerases will enjoy the task of transcribing that gene or nucleic acid sequence. The present invention is primarily concerned with genes and nucleic acid sequences transcribed by eukaryotic RNA polymerase III.

Eukaryotic RNA polymerase III transcribes a limited set of genes comprising 5SRNA, tRNA, 7SL RNA, U6 snRNA and a few other small stable RNAs. To function efficiently, most RNA polymerase III promoters require sequence elements downstream of the +1 transcription start site, within the transcribed region. However, type III RNA polymerase III promoters do not require any intragenic sequence elements to function. In the case of the exemplary U6 snRNA type III RNA polymerase III promoter, efficient expression depends on the presence of upstream sequence elements comprising: a TATA box between nucleotide positions-30 and -24, a proximal sequence element (PSE) between nucleotide positions-66 and -47, and a distal sequence element (DSE) between nucleotide positions-265 and -149. The best characterized type III RNA polymerase III promoters are those associated with the human H1 RNA gene and the U6 snRNA gene.

The term “operator sequence” refers to a DNA sequence recognized by a specific protein or nucleic acid, that upon binding inhibits or prevents transcription from an adjacent operator sequence. For example, the bacterial tet-o operator/repressor system.

The term “packaging”, as used herein refers to the process whereby a nucleic acid is encapsulated in a viral particle.

The term “randomized” or “randomized sequence”, when referring to any nucleic acid sequence, indicates that the nucleotide base appearing at any given position in the sequence said to be randomized can be any one of the four nucleotides occurring naturally in either RNA or DNA, or any homologue thereof, such that a complete set of randomized nucleic acids for a given length will consist of members having every base sequence permutation over the given length. The randomized sequences can be totally randomized (i.e., the probability of finding a base at any position being one in four) or only partially randomized (e.g., the probability of finding a base at any location can be selected at any level between 0 and 100 percent).

Nucleic acid sequence variants can be produced in a number of ways including chemical synthesis of randomized nucleic acid sequences and size selection from randomly cleaved cellular nucleic acids. Usually, the random nucleic acids are chemically synthesized so that the sequences may incorporate any nucleotide at any position. However, if it is desirable to do so, a bias may be deliberately introduced into the randomized sequence. For example, by altering the molar ratios of precursor nucleoside (or deoxynucleoside) triphosphates of the synthesis reaction. A deliberate bias may be desired, for example, to approximate the proportions of individual bases in a given organism, or to affect secondary structure. Thus, the randomized nucleic acid sequence may contain a fully or partially random sequence; or it may contain subportions of conserved sequence incorporated with randomized sequence. Thus, the synthetic process can be designed to allow the formation of any possible combination over the length of the sequence, thereby forming a library of randomized candidate nucleic acids.

The term “restriction site” refers to a DNA sequence that can be recognized and cut by a specific restriction enzyme.

“Terminators” or “termination sequence” refers to those DNA sequences that cause transcription of a nucleic acid sequence to cease. A termination sequence may be recognized intrinsically by the polymerase, or termination may require additional termination factors to be effective. Each of the three eukaryotic polymerase stops synthesizing RNA in response to different termination sequences. Eukaryotic RNA polymerases I and II generally require factors in addition to nucleic acid sequence elements to effect transcription termination. Eukaryotic RNA polymerase III, however, recognizes termination sequences accurately and efficiently in the apparent absence of other factors. In the case of RNA polymerase Type III, simple clusters of four or more thymidine residues serve efficiently as terminators.

The terms “complementary” or “complementarity” refer to polynucleotides (i.e., a sequence of nucleotides) related by base-pairing rules. For example, the sequence “5′-AGT-3′,” is complementary to the sequence “5′-ACT-3′”. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance for methods that depend upon binding between nucleic acids.

A “complementary termination sequence” refers to a nucleic acid sequence that has a nucleotide sequence complementary to a transcription termination sequence of a given promoter.

The term “operably linked” refers to a linkage of polynucleotide elements in a functional relationship. With regard to the present invention, the term “operably linked” refers to a functional linkage between a nucleic acid expression control sequence (such as a promoter, or an array of transcription factor binding sites) and a second nucleic acid sequence, wherein the expression control sequence directs transcription of the nucleic acid corresponding to the second sequence. Thus, a nucleic acid is “operably linked” when it is placed into a functional relationship with another nucleic acid sequence.

Promoters, terminators and control elements “operably linked” to a nucleic acid sequence of interest are capable of affecting the expression of the nucleic acid sequence of interest. The control elements need not be contiguous with the coding sequence, so long as they function to direct the expression thereof. Thus, for example, a promoter or terminator is “operably linked” to a coding sequence if it affects the transcription of the coding sequence.

The phrase “each end operably linked” refers to a relational orientation of a pair of promoter, terminator and/or control elements to a nucleic acid such that both the 5′ and 3′ ends of each single strand of the nucleic acid is operably linked to a promoter, terminator and/or control elements allowing for transcription of the respective strands of the nucleic acid. Transcription of such a construct produces two complementary RNA transcripts. The complementary RNA molecules produced can base pair with one another to form a dsRNA molecule.

The phrase “oriented to initiate” refers to the relationship of a promoter sequence with respect to a nucleic acid sequence of interest. Promoters are “oriented to initiate” transcription when they are operably linked to a nucleic acid sequence of interest in such a way that the promoter is capable of causing transcription of the nucleic acid sequence of interest to begin when appropriate inducing signals are transmitted to the system comprising the promoter.

The term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vectors, as well as viral vectors.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the recombinant expression cassette portion of the expression vector includes a nucleic acid to be transcribed, and a promoter.

A “DNA expression cassette” refers to a DNA sequence capable of directing expression of a nucleic acid in cells. A “DNA expression cassette” comprises a promoter, operably linked to a nucleic acid of interest, which is further operably linked to a termination sequence. An “siRNA gene” is a DNA expression cassette capable of expressing siRNA.

The term “host cell” refers to a cell that contains an expression vector and supports the replication or expression of the expression vector. A host cell can be prokaryotic cells such as E. coli, or eukaryotic cells such as yeast, insect, or mammalian cells.

A “viral particle” refers to an intact virus comprising a nucleic acid core a proteinaceous capsid and, in some cases, an outer envelope.

The term “viral transduction system” refers to the use of viral vectors to introduce an exogenous nucleic acid into a cell. Viral transduction systems can be DNA or RNA-based, but are generally incorporated into the infected cell in a DNA form, either as an integrated part of the cellular genome, or as an episomal genetic element.

### DETAILED DESCRIPTION OF PREFERRED EMBODIMENTS

I. Expression Cassettes

The present invention is directed to novel expression cassette constructs and methods for making the same so as to express siRNAs. The expression cassettes in accordance with the invention comprise a double-stranded nucleic acid which when transcribed will produce an siRNA of from 16 to 25 bases long, more preferably between 17 and 23 bases long, and most preferably between 18 and 21 bases long. siRNAs of this length have been reported to silence both endogenous and heterologous genes without triggering interferon responses that are intrinsically sequence non-specific. Elbashir, Sayda M. et al., Nature, 411:494-498 (2001); Tuschl, Thomas, Nature Biotechnology, 20:446-448 (2002); Paul, Cynthia P., et al., Nature Biotechnology, 20:505-508 (2002).

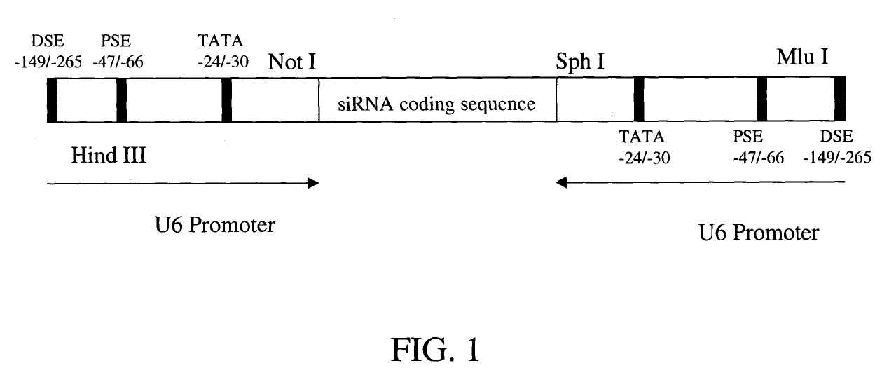

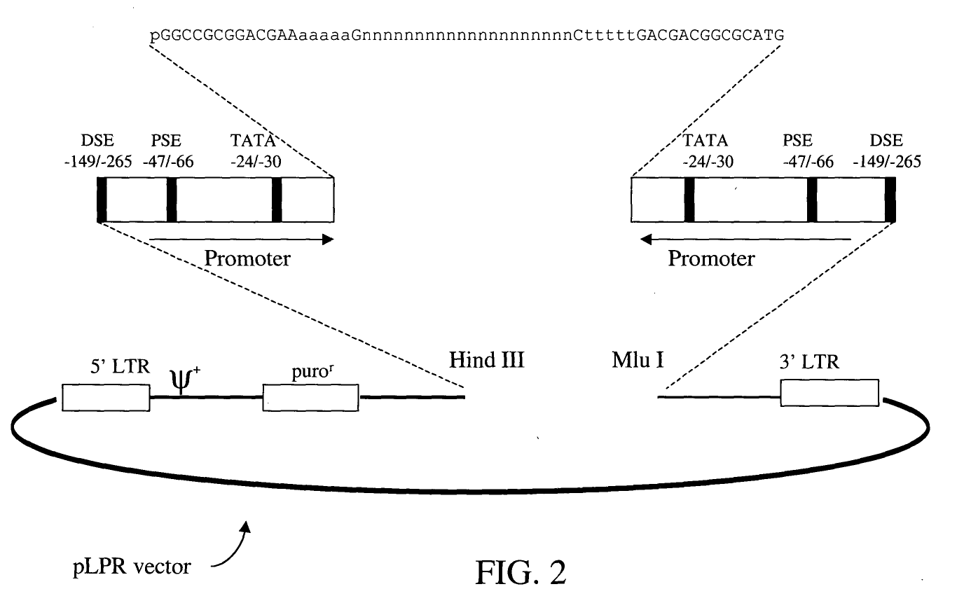

The nucleic acid of the expression cassette in accordance with the invention is situated between a pair of modified promoters of a dual promoter expression system as depicted schematically in FIGS. 1 and 2. As shall be explained in greater detail below, the dual promoter expression system allows for transcription of one strand of the coding sequence to initiate from one of the two promoters and transcription of the opposing strand of the coding sequence to initiate from the other promoter. FIGS. 1 and 2 show insertion of the coding sequence between the opposing promoters facilitated by the Not I and Sph I restriction sites, although those of skill in the art will recognize that other restriction sites and methods can be used to accomplish insertion of the dsRNA coding sequence between the promoters of the system.

If the nucleic acid is of a known sequence, it may be isolated from a biological source such as RNA, cDNA, genomic DNA, or a hybrid of these. More typically, one strand of the nucleic acid will be chemically synthesized using techniques well known in the art. This is particularly true when the nucleic acid comprising the coding sequence comprises a random sequence. In such event, the randomized sequence will preferably be flanked by nucleotides of known sequence, between 4 and 24 bases long, more preferably 5-20 bases long. The complementary strand of the nucleic acid is synthesized, preferably enzymatically, after the single strand bearing the coding sequence for the dsRNA is ligated between the oppositely orientated promoters.

FIG. 2 shows an embodiment of the invention in which the coding sequence is randomized. For illustrative purposes, this randomized sequence is shown with a G at its 5′ end and a C at its 3′ end. The 5′ G is the first transcribed nucleotide of the RNA transcript produced from the strand depicted in the figure. The 3′ C is the complement to the first base of the complementary strand (not shown) which will be transcribed by the opposing promoter. FIG. 2 also depicts the expression cassette being incorporated into a preferred retroviral vector, pLPR. Incorporation is facilitated by a Hind III and a Mlu I site in the vector, with corresponding sites in the regions flanking the expression cassette promoters. The expression cassette in inserted into the vector at a position between the two LTRs, a region shared with the selectable marker puro^{r}, or at a position within the 3′ LTR (not shown).

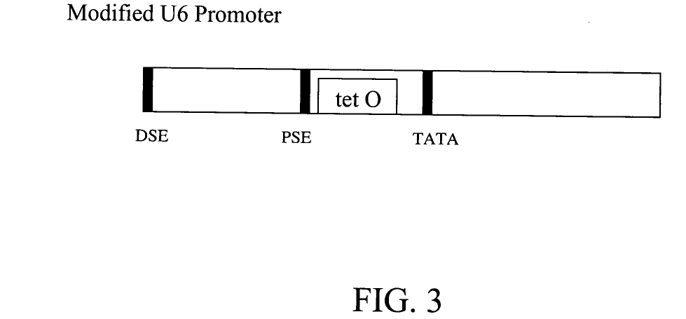

As illustrated in FIG. 3, the promoters of the dual promoter expression system may be modified to include transcriptional regulatory sequences. Such sequences allow for differential expression from the expression cassette, controlled by the cellular environment or cell type. FIG. 3 illustrates an exemplary regulatory sequence, the Tet-o operator. As depicted in the figure, the operator sequence is positioned 5′ to the TATA box, although other positions are possible. Regulatory sequences may be engineered by those skilled in the art to work with any promoter compatible with the dual promoter expression system using the methods described herein. It should also be noted that regulatory sequences affecting expression from the promoters of the present invention need not be located within the promoter sequence itself.

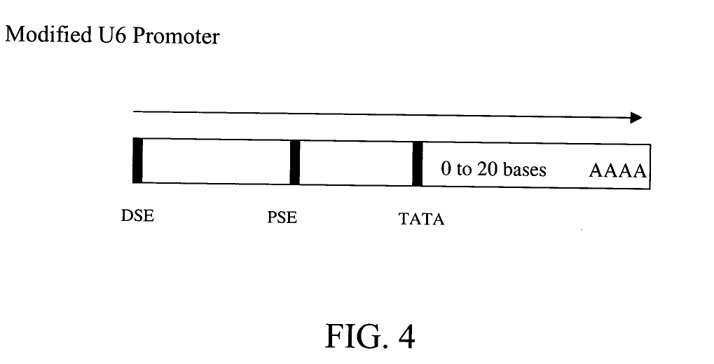

FIG. 4 illustrates another unique aspect of the promoters of the present invention, the ability to incorporate directly into the promoter a sequence complementary to the termination sequence of the companion promoter of the dual promoter expression system. By way of example, FIG. 4 shows a human U6 promoter modified by substituting four adenylyl residues for the original four nucleotides of the 3′ end of the promoter sequence. Four adenylyl residues are shown in this example as four thymidyl residues are an effective termination sequence for the companion U6 promoter. It is to be noted however that any of the last 25 bases located at the 3′ end of the promoter sequence may be substituted so as to be complementary to the termination sequence of the companion promoter. Requirements for promoter-internalized termination sequences of this nature are that the sequence be no more than 25 bases long and that it does not prevent transcriptional initiation from the promoter.

The expression cassettes of the present invention can therefore be described structurally as a coding sequence flanked by two promoters in opposite orientation such that one promoter initiates transcription of the “sense” strand of the coding sequence while the other promoter initiates transcription of the “antisense” strand. Each promoter contains a sequence at its 3′ end that is complementary to the termination sequence of the opposing promoter.

Functionally, the expression cassette allows transcription of both strands of a common DNA molecule, producing a dsRNA. Typically, at least the first two bases of each termination sequence are also transcribed, such that these dsRNAs have 3′ overhangs which can be of any sequence, but preferably consist of two thymidyl residues.

II. General Recombinant Methods

The expression cassettes and vectors of the present invention may be constructed utilizing standard techniques that are well known to those of ordinary skill in the art (Sambrook, J., Fritsch, E. F., and Maniatus, T., Molecular Cloning, A Laboratory Manual 2nd ed. (1989); Gelvin, S. B., Schilperoort, R. A., Varma, D. P. S., eds. Plant Molecular Biology Manual (1990)).

In preparing the expression cassettes of the present invention, the various DNA sequences may normally be inserted or substituted into a bacterial plasmid. Any convenient plasmid may be employed, which will be characterized by having a bacterial replication system, a marker which allows for selection of transformed bacteria and generally one or more unique, conveniently located restriction sites. These plasmids, referred to as vectors, may include such vectors as pACYC184, pACYC177, pBR322, pUC9, or pBluescript II (KS or SK), the particular plasmid being chosen based on the nature of the markers, the availability of convenient restriction sites, copy number, and the like. Thus, the sequence may be inserted into the vector at an appropriate restriction site(s), the resulting plasmid used to transform the E. coli host, the E. coli grown in an appropriate nutrient medium and the cells harvested and lysed and the plasmid recovered. One then defines a strategy that allows for the stepwise combination of the different fragments.

It will be appreciated that the practice of the present invention involves generating alterations in nucleic acid sequences, which may be accomplished utilizing any of the methods known to one skilled in the art, including site-specific mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Using these techniques, it is possible to insert or delete, at will, a polynucleotide of any length into an expression cassette of the present invention.

The practice of the present invention also involves chemical synthesis of linear oligonucleotides which may be carried out utilizing techniques well known in the art. The synthesis method selected will depend on various factors including the length of the desired oligonucleotide and such choice is within the skill of the ordinary artisan. Oligonucleotides are typically synthesized chemically according to the solid phase phosphoramidite triester method described by Beaucage and Caruthers, Tetrahedron Letts., 22(20):1859-1862 (1981), e.g., using an automated synthesizer, as described in Needham-VanDevanter et al., Nucleic Acids Res., 12:6159-6168 (1984). Oligonucleotides can also be custom made and ordered from a variety of commercial sources known to persons of skill in the art.

Synthetic linear oligonucleotides may be purified by polyacrylamide gel electrophoresis, or by any of a number of chromatographic methods, including gel chromatography and high pressure liquid chromatography. The sequence of the synthetic oligonucleotides can be verified using the chemical degradation method of Maxam and Gilbert in Grossman and Moldave (eds.) Academic Press, New York, Methods in Enzymology, 65:499-560(1980). If modified bases are incorporated into the oligonucleotide, and particularly if modified phosphodiester linkages are used, then the synthetic procedures are altered as needed according to known procedures. In this regard, Uhlmann, et al., Chemical Reviews, 90:543-584 (1990) provide references and outline procedures for making oligonucleotides with modified bases and modified phosphodiester linkages. Sequences of short oligonucleotides can also be analyzed by laser desorption mass spectroscopy or by fast atom bombardment (McNeal, et al., J. Am. Chem. Soc., 104:976 (1982); Viari, et al., Biomed. Enciron. Mass Spectrom., 14:83 (1987); Grotjahn et al., Nuc. Acid Res., 10:4671 (1982)).

As indicated, the second strand of the coding nucleic acid of the invention typically is synthesized enzymatically. Enzymatic methods for DNA oligonucleotide synthesis frequently employ Klenow, T7, T4, Taq or E. coli DNA polymerase as described in Sambrook et al., in Molecular Cloning: A Laboratory Manual, Cold Spring Harbor, N.Y. (1989). Enzymatic methods for RNA oligonucleotide synthesis frequently employ SP6, T3 or T7 RNA polymerase as described in Sambrook et al., (1989). Reverse transcriptase can also be used to synthesize DNA from RNA or DNA templates (Sambrook et al., 1989)

Linear oligonucleotides may also be prepared by polymerase chain reaction (PCR) techniques as described, for example, by Saiki et al., Science, 239:487 (1988). In vitro amplification techniques suitable for amplifying nucleotide sequences are also well known in the art. Examples of such techniques including the polymerase chain reaction (PCR), the ligase chain reaction (LCR), Qβ-replicase amplification and other RNA polymerase mediated techniques (e.g., NASBA) are found in Berger, Sambrook, and Ausubel, as well as Mullis et al., (1987) U.S. Pat. No. 4,683,202; PCR Protocols A Guide to Methods and Applications (Innis et al., eds) Academic Press Inc., San Diego, Calif. (1990) (Innis); Arnheim & Levinson (Oct. 1, 1990) C&EN 36-47; The Journal Of NIH Research, 3:81-94 (1991); (Kwoh et al., (1989) Proc. Natl. Acad. Sci. USA, 86:1173; Guatelli et al., Proc. Natl. Acad. Sci. USA, 87:1874 (1990); Lomell et al., J. Clin. Chem, 35:1826 (1989); Landegren et al., Science, 241:1077-1080 (1988); Van Brunt, Biotechnology, 8:291-294 (1990); Wu and Wallace, Gene, 4:560 (1989); Barringer et al., Gene, 89:117 (1990), and Sooknanan and Malek, Biotechnology, 13:563-564 (1995). Improved methods of cloning in vitro amplified nucleic acids are described in Wallace et al., U.S. Pat. No. 5,426,039.

III. Coding Sequences

The coding region for the expression cassettes of the present invention are the sequences transcribed to produce dsRNAs. These dsRNA coding sequences can be isolated from genomic or cDNA libraries using standard techniques well known in the art. (Gubler & Hoffman, Gene, 25:263-269 (1983); Sambrook et al., Molecular Cloning: A Laboratory Manual; Cold Spring Harbor Laboratory, Cold Spring Harbor, N.Y. (1989); Ausubel et al.).

Alternatively, the dsRNA coding sequences can be synthesized chemically. For randomized dsRNA coding sequences, all four bases are included in those synthesis cycles where randomized sequences are desired. Preferably, the randomized dsRNA coding sequences are flanked by nucleotides of known sequence. These become the 3′ end sequences for the promoters of the dual promoter system when the randomized dsRNA coding sequences are ligated into the expression cassette. Flanking sequences to the randomized dsRNA coding sequences provide a number of useful purposes: First, these sequences provide a convenient means for ligating the randomized dsRNA coding sequence into the expression cassette in the correct orientation. By having a different hybridization sequence (usually a restriction site sequence) for each of the dual promoters and complementary sequences to these hybridization sequences at the appropriate ends of the randomized dsRNA coding sequence, the latter sequence can be directionally orientated in the cassette. Second, the known sequences flanking the randomized dsRNA coding sequence provide a means for engineering genetic and cloning elements into the dual promoters of the invention. These elements include, but are not limited to, transcriptional termination sequences, operator sequences and restriction sites. If however these flanking sequences are undesired, they can be removed by processes known in the art, such as exonuclease III-mediated deletion.

For dsRNA's of known sequence, both the “sense” and “antisense” strands can be synthesized chemically with appropriate overhanging ends, hybridized to each other, and ligated directly into the vector between the opposing promoters.

IV. Promoters

As already explained, the present invention comprises a novel dual promoter system that allows simultaneous transcription of both the “sense” and “antisense” strands of a sequence encoding a dsRNA. The particular promoters chosen for use in the expression cassettes of the present invention will depend upon which organism or cell type is to be targeted by the dsRNA encoded in the expression cassette. For example, if plant cells are to be the target for the dsRNA, then plant promoters should be used. If mammalian cells are to be the target for the dsRNA, then mammalian promoters should be used. The promoters can be constitutive, inducible, or cell dependent, depending on the application and result desired. The promoters do not have to be the same, although they can be. Promoters can be of different types, isolated from different genes, be differentially regulated or differ by as little as one base.

Pol III promoters are preferred for the expressions cassettes of the present invention. The type I and type II pol III promoters (e.g., the promoters for tRNA genes and the adenovirus VA genes) require elements located downstream of the transcription start site (i.e., within the associated structural gene). In contrast, the type III pol III promoters (e.g., the U6 small nuclear (sn) RNA and the H1 RNA promoters) lack any requirement for intragenic promoter elements. They contain all of the cis-acting promoter elements upstream of the transcription start site, including a traditional TATA box (Mattaj et al., Cell, 55:435-442 (1988)), a proximal sequence element (PSE) and in some circumstances a distal sequence element (DSE; Gupta and Reddy, Nucleic Acids Res., 19:2073-2075 (1991)). For certain applications, the type III promoters may be preferred, since the absence of intragenic promoter elements allows for greater flexibility when designing the coding region of the cassette. For other applications where additional considerations may be paramount (e.g., cytoplasmic localization of the siRNAs), other pol III promoters may be preferred. Both type II and type III pol III promoters have been used to express siRNAs (Brummelkamp et al. (2002) Science 296: 550-553; Paddison et al. (2002), Genes and Development 16: 948-958; Miyagishi and Taira (2002), Nature Biotechnology, 20:497-500; Lee et al., Ibid.:500-505; Paul et al., Ibidl.: 505-508; Kawasaki and Taira (2003), Nucleic Acids Res. 31:700-707).

The promoters in accordance with the invention preferably will not have a requirement for a particular nucleotide at the transcription start-point, thereby optimizing flexibility in designing the dsRNA coding sequence, although some specificity is tolerable, including a specific requirement for a G or A at the first position by some polymerases (see, e.g., FIG. 2).

In the construction of heterologous promoter/reading frame combinations, the promoter is preferably positioned about the same distance from the heterologous transcription start site as it is from the transcription start site in its natural setting, although some variation in this distance may be accommodated without loss of promoter function under certain conditions.

Several methods for isolation of promoters are known. For instance, the full length of a promoter sequence may be isolated if a portion of the promoter or the corresponding gene sequence is known. One skilled in the art will recognize that a variety of small or large insert genomic DNA libraries may be screened using hybridization or polymerase chain reaction (PCR) technology to identify library clones containing the desired sequence. Typically, the desired sequence may be used as a hybridization probe to identify individual library clones containing the known sequence. Alternatively, PCR primers based on the known sequence may be designed and used in conjunction with other primers to amplify sequences adjacent to the known DNA polynucleotide sequence. Library clones containing adjacent DNA sequences may thereby be identified. Restriction mapping and hybridization analysis of the resulting library clones' DNA inserts allows for identification of the DNA sequences adjacent to the known DNA polynucleotide sequence. Thus, promoters may be isolated if only a portion of a promoter sequence is known.

Promoter regions of the invention typically are engineered to contain restriction sequences, both internal and flanking, to aid in the cloning process.

Transcription Terminators

Transcription terminators allow for the efficient cessation of transcription, once the coding sequence of the expression cassette has been transcribed. Transcription terminators of the present invention preferably have a minimal structural complexity and do not signal post-transcriptional processing events, such as polyadenylation. A minimal structure is preferred as the transcriptional terminators are ideally located between the coding sequence for the dsRNA and the promoter sequence for transcribing the opposing nucleotide strand, most preferably forming part of the 3′ end of the promoter sequence for transcribing the opposing nucleotide strand. Post transcriptional processing is not preferred as the desired product formed by the novel promoter system of the present invention is a dsRNA with 3′ overhangs of at least 2 nucleotides. Tuschl, Thomas, Nature Biotechnology, 20:446-448 (2002); Miyagishi and Taira, Nature Biotechnology, 20:497-500 (2002). Accordingly, preferable transcriptional terminators comprise between 4 and 25 nucleic acids, of which at least four consecutive nucleic acids are thymidyl residues (see Miyagishi and Taira, supra). Preferable terminators include the minimal termination sequence for pol III, type III polymerases, a sequence of four consecutive thymidyl residues. The complementary sequence for such a termination sequence is shown in FIG. 4, in this instance engineered in a preferred position at the 3′ distal end of a promoter of the present invention. Referring to FIG. 4, the complementary terminator sequence is not limited to four adenylyl residues, even when engineered into the promoter as described herein. Any of the 20 bases of the region immediately 5′ to the four adenylyl region can be substituted to accommodate a larger termination sequence. Restriction sites may also be included in this region to ease incorporation of such substitutions by methods well known in the art (Sambrook et al., supra; Ausubel et al., supra).

Generally, any termination sequence capable of terminating transcription of the polymerase reaction initiated at the companion promoter of the expression cassette can be used. Suitable 3′ termination sequences can be isolated from genomic libraries, through amplification techniques using oligonucleotide primers, or can be constructed chemically, as described above.

Engineering Promoter/Terminator Combinations

A feature of the present invention is the functional combination of promoter/terminator sequences that are capable of initiating transcription of one strand, while concomitantly terminating transcription of the complementary strand. Promoter/terminator sequences of the present invention incorporate a transcriptional termination sequence into the 3′ distal end of a functional promoter. Incorporation of the terminator is done in a manner that does not disturb the transcriptional start site for the promoter, a process that usually requires deletion of sequence from the native promoter to accommodate the terminator sequence.

Engineering the terminator into the promoter sequence can be accomplished by any of the techniques well known in the art. For example, site-directed mutagenesis can be performed to create a restriction site that has a single-stranded end when cleaved, at the desired position in the 3′ region of the promoter. (see, e.g., Adelman et al., DNA, 2:183, (1983)).

Alternatively, the synthetic nucleotide having a complementary single-stranded end to that generated by restriction of the engineered promoter site and comprising the sequence for the desired terminator can be synthesized as the known flanking sequence for the dsRNA coding sequence described herein. In this alternative, hybridizing and ligating the complementary ends also positions the dsRNA coding sequence between the promoters. The complementary strand for the coding sequence is then synthesized, preferably enzymatically as described supra.

The termination sequence can also be engineered into the promoter in a manner producing a 3′ blunt end to the promoter, whereby transcription preferably starts at the first nucleic acid of a nucleotide ligated to the blunt end. In this circumstance, the coding sequence for the dsRNA can simply be blunt-end ligated into position between the two promoters of the invention. (see e.g., Sambrook et al., supra; Ausubel et al., supra).

One or more restriction sites can also be engineered into the 3′ end of the promoter, preferably between the terminator sequence and the TATA box. Engineered restriction sites ease cloning manipulations and allow for easy isolation of the coding sequence for the dsRNA. The combined length of the termination sequence and restriction site sequence should be between 4 and 25 bases in size, preferably between 4 and 20 bases, most preferably between 5 and 16 bases long. Of course other genetic elements can be substituted for or included with the engineered restriction site, provided that the stated nucleotide sequence length is met.

Expression Control Elements

Several embodiments of the present invention comprise expression control elements that function to regulate initiation of transcription as well as the rate at which transcription progresses. These sequences control such aspects of expression as plasmid copy number, recombination characteristics (e.g., site specific or promiscuous integration into the cellular genome) and promoter activity. Expression control sequences are important as they determine whether the expression cassettes of the present invention are stably or transiently integrated into a cell and at what levels the dsRNA encoded in the expression cassette will be expressed once the expression cassette is integrated.

One such control element is a cis-acting operator sequence recognized by a trans-acting factor(s). This operator sequence comprises one or more nucleotide sequences that may be engineered into the promoter itself, or into the vector containing the promoter at a suitable position that allows for regulation of polymerase activity from the promoter when trans-acting factors recognizing the operator sequence are present. Trans-acting factors may be encoded into the same vector or chromosome as the expression cassette of the invention, or in other vectors or chromosomes.

Operator sequences recognized by trans-acting factors confer inducible characteristics upon expression from the promoters of the dual promoter system described herein. Induction of expression can be accomplished by a variety of means, depending on the particular operator system employed. For example, some operators systems confer tissue-specific expression characteristics to the promoters. Other operators are activated by small molecules and hormones. Exemplary operator systems include the ecdysone/glucocorticoid response element (GRE) (Invitrogen, Carlsbad, Calif.); the Tet operon (Clontech, Palo Alto, Calif.; Invitrogen, Carlsbad, Calif.); and the Lac operon (Hu and Davidson (1987) Cell, 48:555-556). Additional regulatory sequences are described, for example, in Goeddel, Gene Expression Technology: Methods in Enzymology, 185, Academic Press, San Diego, Calif. (1990). Other illustrative mammalian expression control sequences are obtained from the SV-40 promoter (Science, 222:524-527 (1983)), the CMV I.E. Promoter (Proc. Natl. Acad. Sci., 81:659-663 (1984)) or the metallothionein promoter (Nature, 296:39-42 (1982)).

A preferred expression control element (operator sequence) for use with the expression cassettes of the present invention is the tetracycline (tet) operator sequence (tet O). As depicted in FIG. 3, tet O may be engineered into a modified U6 snRNA promoter for use with the present invention. When tet O is bound by a tetracycline-sensitive trans-acting protein (tetracycline repressor, Tet R), transcriptional initiation at the promoter is prevented. When tet O is not bound by Tet R, transcription from the promoter can proceed, allowing expression of the coding sequence operably linked to it (see: Ohkawa and Taira, Human Gene therapy, 11:577-585 (2000); van de Wetering, EMBO Reports, 4:609-615 (2003).

V. Recombinant Vectors

Another aspect of the invention pertains to vectors containing the expression cassettes of the invention. Certain types of vectors allow the expression cassettes of the present invention to be amplified. Other types of vectors are necessary for efficient introduction of the expression cassettes to cells and their stable expression once introduced. Any vector capable of accepting a DNA expression cassette of the present invention is contemplated as a suitable recombinant vector for the purposes of the invention. The vector may be any circular or linear length of DNA that either integrates into the host genome or is maintained in episomal form. Vectors may require additional manipulation or particular conditions to be efficiently incorporated into a host cell (e.g., many expression plasmids), or can be part of a self-integrating, cell specific system (e.g., a recombinant virus).

Each vector system has advantages and disadvantages, which relate, among others, to host cell range, intracellular location, level and duration of dsRNA expression, and ease of scale-up/purification. Optimal delivery systems are characterized by: 1) broad host range; 2) high titer/μg DNA; 3) stable expression; 4) non-toxic to host cells; 5) no replication in host cells; 6) ideally no viral gene expression; 7) stable transmission to daughter cells; 8) high rescue yield; and 9) lack of subsequent replication-competent virus that may interfere with subsequent analysis. Choice of vector may also depend on the intended application.

Episomal vectors generally have extrachromosomal replicators that, in addition to their origin function, encode functions that assure equal distribution of replicated molecules between daughter cells at cell division. In higher organisms, different mechanisms exist for partitioning of extrachromosomal replicators. For example, artificial (ARS-containing) plasmids in yeast utilize chromosomal centromeres as extrachromasomal replicators (Struhl et. al., Proc. Natl. Acad. Sci USA, 76:1035-1039 (1979)). In metazoan cells, one well studied example of a stable extrachromosomal replicator—is the latent origin oriP from Epstein-Barr Virus (EBV) (see Yates et al., Proc. Natl. Acad. Sci USA, 81:3806-3810 (1984); Yates et al., Nature, 313:812-815 (1985), and Krysan et al., MoL Cell. Biol., 9:1026-1033 (1989)).

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Certain vectors, “expression vectors”, are capable of directing the expression of genes. Any expression vector comprising an expression cassette of the present invention qualifies as an expression cassette of the present invention. In general, expression vectors of utility in recombinant DNA techniques often are in the form of plasmids. However, preferred vector systems of the present invention are viral vectors, e.g., replication defective retroviruses, lentiviruses, adenoviruses and adeno-associated viruses, baculovirus, CaMV and the like, which are discussed in greater detail below.

As an example, a expression vector construct for use in a mammalian target cell in accordance with the present invention may include:

1. A DNA expression cassette, as described supra, including a dual promoter system that functions in the selected target cell, such as one derived from the mammalian U6 gene (an RNA polymerase III promoter) which directs transcription in mammalian cells.

2. A mammalian origin of replication (optional) that allows episomal (non-integrative) replication, such as the origin of replication derived from the Epstein-Barr virus.

3. An origin of replication functional in bacterial cells for producing required quantities of the DNA expression cassettes of the present invention, such as the origin of replication derived from the pBR322 plasmid.

4. A mammalian selection marker (optional), such as neomycin or hygromycin resistance, which permits selection of mammalian cells that are transfected/transduced with the construct.

5. A bacterial antibiotic resistance marker, such as kanamycin or ampicillin resistance, which permits the selection of bacterial cells that are transformed with the plasmid vector.

Examples of suitable E. coli expression vectors that can be engineered to accept a DNA expression cassette of the present invention include pTrc (Amann et al., Gene, 69:301-315 (1988)) and pBluescript (Stratagene, San Diego, Calif.). Examples of vectors for expression in yeast S. cerevisiae include pYepSec1 (Baldari et al., EMBO J., 6:229-234 (1987)), pMFa (Kurjan and Herskowitz, Cell, 30:933-943 (1982)), pJRY88 (Schultz et al., Gene, 54:113-123 (1987)), pYES2 (Invitrogen, Carlsbad, Calif.), and pPicZ (Invitrogen, Carlsbad, Calif.). Baculovirus vectors are the preferred system for expression of dsRNAs in cultured insect cells (e.g., Sf9 cells see, U.S. Pat. No. 4,745,051) and include the pAc series (Smith et al., Mol. Cell Biol., 3:2156-2165 (1983)), the pVL series (Lucklow and Summers, Virology, 170:31-39 (1989)) and pBlueBac (available from Invitrogen, San Diego). For other suitable expression systems for both prokaryotic and eukaryotic cells see chapters 16 and 17 of Sambrook et al., supra. Preferred mammalian vectors are generally of viral origin and are discussed in detail below.

Mammalian Viral Vectors

Infection of cells with a viral vector is a preferred method for introducing expression cassettes of the present invention into cells. The viral vector approach has the advantage that a large proportion of cells receive the expression cassette, which can obviate the need for selection of cells that have been successfully transfected. Exemplary mammalian viral vector systems include retroviral vectors, lentiviral vectors, adenoviral vectors, adeno-associated type 1 (“AAV-1”) or adeno-associated type 2 (“AAV-2”) vectors, hepatitis delta vectors, live, attenuated delta viruses and herpes viral vectors.

(a) Retroviruses

Retroviruses are RNA viruses that are useful for stably incorporating genetic information into the host cell genome. When a retrovirus infects cells, their RNA genomes are converted to a dsDNA form (by the viral enzyme reverse transcriptase). The viral DNA is efficiently integrated into the host genome, where it permanently resides, replicating along with host DNA at each cell division. The integrated provirus steadily produces viral RNA from a strong promoter located at the end of the genome (in a sequence called the long terminal repeat or LTR). This viral RNA serves both as mRNA for the production of viral proteins and as genomic RNA for new viruses. Viruses are assembled in the cytoplasm and bud from the cell membrane, usually with little effect on the cell's health. Thus, the retrovirus genome becomes a permanent part of the host cell genome, and any foreign gene placed in a retrovirus ought to be expressed in the cells indefinitely. Retroviruses are therefore attractive vectors because they can permanently express a foreign gene in cells. Most or possibly all regions of the host genome are accessible to retroviral integration (Withers-Ward et al., Genes Dev., 8:1473-1487 (1994)). Moreover, they can infect virtually every type of mammalian cell, making them exceptionally versatile.

Retroviral vector particles are prepared by recombinantly inserting an expression cassette of the present invention into a retroviral vector and packaging the vector with retroviral proteins by use of a packaging cell line or by co-transfecting non-packaging cell lines with the retroviral vector and additional vectors that express retroviral proteins. The resultant retroviral vector particle is generally incapable of replication in the host cell and is capable of integrating into the host cell genome as a proviral sequence containing the expression cassette containing a nucleic acid encoding a dsRNA. As a result, the host cell produces the dsRNA encoded by the nucleic acid of the expression cassette. A useful retroviral construct for introducing expression cassettes of the present invention is depicted in FIG. 2. The figure illustrates the positioning of the expression cassette (between the pair of long terminal repeats) and the presence of a selectable marker, in this case puro^{r}. The expression cassette may also be located within the 3′ LTR (see: Barton and Medzhitov (2002) Proc. Natl. Acad. Sci. USA 99: 14943-14945;Gervaix et al. (1997) J. Virol. 71: 3048-3053).

Packaging cell lines are generally used to prepare the retroviral vector particles. A packaging cell line is a genetically constructed mammalian tissue culture cell line that produces the necessary viral structural proteins required for packaging, but which is incapable of producing infectious virions. Retroviral vectors, on the other hand, lack the structural genes but have the nucleic acid sequences necessary for packaging. To prepare a packaging cell line, an infectious clone of a desired retrovirus, in which the packaging site has been deleted, is constructed. Cells comprising this construct will express all structural proteins but the introduced DNA will be incapable of being packaged. Alternatively, packaging cell lines can be produced by introducing into a cell line one or more expression plasmids encoding the appropriate core and envelope proteins. In these cells, the gag, pol, and env genes can be derived from the same or different retroviruses.

A number of packaging cell lines suitable for the present invention are available in the prior art. Examples of these cell lines include Crip, GPE86, PA317 and PG13. See Miller et al., J. Virol., 65:2220-2224 (1991), which is incorporated herein by reference. Examples of other packaging cell lines are described in Cone and Mulligan, Proceedings of the National Academy of Sciences, U.S.A., 81:6349-6353 (1984) and in Danos and Mulligan, Proceedings of the National Academy of Sciences, U.S.A., 85:6460-6464 (1988); Eglitis et al., Biotechniques, 6:608-614 (1988); Miller et al., Biotechniques, 7:981-990 (1989), also all incorporated herein by reference. Amphotropic or xenotropic envelope proteins, such as those produced by PA317 and GPX packaging cell lines may also be used to package the retroviral vectors.

Defective retroviruses are well characterized for use in gene transfer to mammalian cells (for a review see Miller, A. D., Blood, 76:271 (1990)). A recombinant retrovirus can be constructed having a nucleic acid encoding an expression cassette of the present invention inserted into the retroviral genome. Additionally, portions of the retroviral genome can be removed to render the retrovirus replication defective. The replication defective retrovirus is then packaged into virions that can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals.

Examples of retroviruses encompassed by the present invention include pLJ, pZIP, pWE and pEM which are well known to those skilled in the art. Examples of suitable packaging virus lines include Ψ Crip, Ψ Cre, Ψ 2, and Ψ Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al., Science, 230:1395-1398 (1985); Danos and Mulligan, Proc. Natl. Acad. Sci. USA, 85:6460-6464 (1988); Wilson et al., Proc. Natl. Acad. Sci. USA, 85:3014-3018 (1988); Armentano et al., Proc. Natl. Acad. Sci. USA, 87:6141-6145 (1990); Huber et al., Proc. Natl. Acad. Sci. USA, 88:8039-8043 (1991); Ferry et al., Proc. Natl. Acad. Sci. USA, 88:8377-8381 (1991); Chowdhury et al., Science, 254:1802-1805 (1991); van Beusechem et al., Proc. Natl. Acad. Sci. USA, 89:7640-7644 (1992); Kay et al., Human Gene Therapy, 3:641-647 (1992); Dai et al., Proc. Natl. Acad. Sci. USA, 89:10892-10895 (1992); Hwu et al., J. Immunol., 150:4:104-115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573; EPA 0 178 220; U.S. Pat. No. 4,405,712; Gilboa, Biotechniques, 4:504-512 (1986); Mann et al., Cell, 33:153-159 (1983); Cone and Mulligan, Proc. Natl. Acad. Sci. USA, 81:6349-6353 (1984); Eglitis et al., Biotechniques 6:608-614 (1988); Miller et al., Biotechniques, 7:981-990 (1989); Miller, Nature (1992), supra; Mulligan, Science, 260:926-932 (1993); and Gould et al., and International Patent Application No. WO 92/07943 entitled “Retroviral Vectors Useful in Gene Therapy.”). The teachings of these patents and publications are incorporated herein by reference.

(b) Adenoviruses

The genome of an adenovirus can be manipulated such that it encodes an expression cassette of the present invention, but is inactivated in terms of its ability to replicate in a normal lytic viral life cycle. See for example Berkner et al., BioTechniques, 6:616 (1988); Rosenfeld et al., Science, 252:431-434 (1991); and Rosenfeld et al., Cell, 68:143-155 (1992). Suitable adenoviral vectors derived from the adenovirus strain Ad type 5 d1324 or other strains of adenovirus (e.g., Adz, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al., Proc. Natl. Acad. Sci. USA, 89):6482-6486 (1992)), hepatocytes (Herz and Gerard, Proc. Natl. Acad. Sci. USA, 90:2812-2816 (1993)) and muscle cells (Quantin et al., Proc. Natl. Acad. Sci. USA, 89:2581-2584 (1992)).

(c) Adeno-Associated Viruses

Adeno-associated virus (AAV) is a naturally occurring defective virus that requires another virus, such as an adenovirus or a herpes virus, as a helper virus for efficient replication and a productive life cycle. (For a review see Muzyczka et al., Curr. Topics in Micro. and Immunol., 158:97-129 (1992)). It exhibits a high frequency of stable integration (see for example Flotte et al., Am. J Respir. Cell. Mol. Biol., 7:349-356 (1992); Samulski et al., J. Virol., 63:3822-3828 (1989); and McLaughlin et al., J. Virol, 62:1963-1973 (1989); Flotte, et al., Gene Ther., 2:29-37 (1995); Zeitlin, et al., Gene Ther., 2:623-31 (1995); Baudard, et al., Hum. Gene Ther., 7:1309-22 (1996); which are hereby incorporated by reference). Vectors containing as little as 300 base pairs of AAV can be packaged and can integrate. Space for exogenous nucleic acid is limited to about 4.5 kb, well in excess of the overall size of the expression vectors of the invention. An AAV vector, such as that described in Tratschin et al., Mol. Cell. Biol.,5:3251-3260 (1985) can be used to introduce the expression vector into cells. A variety of nucleic acids have been introduced into different cell types using AAV vectors (see for example Hermonat et al., Proc. Natl. Acad. Sci. USA, 81:6466-6470 (1984); Tratschin et al., Mol. Cell. Biol., 4:2072-2081 (1985); Wondisford et al., Mol. Endocrinol., 2:32-39 (1988); Tratschin et al., J. Virol., 51:611-619 (1984); and Flotte et al., J. Biol. Chem., 268:3781-3790 (1993)).

Once a cell or cells have been selected and shown to contain a dsRNA coding sequence of interest, the entire dsRNA expression cassette can be easily “rescued” from the host cell genome and amplified by introduction of the AAV viral proteins and wild type adenovirus (Hermonat. and Muzyczka, PNAS. USA, 81:6466-6470 (1984); Tratschin. et al., Mol. Cell. Biol., 5:3251-3260 (1985); Samulski et al., PNAS USA, 79:2077-2081 (1982); Tratschin et al., Mol. Cell. Biol., 5:3251-3260 (1985)). This makes isolation, purification and identification of selected dsRNA's considerably easier than other molecular biology techniques.

(d) Lentiviruses

The expression cassettes of the present invention may also be incorporated into lentiviral vectors. In this regard, see: Qin et al. (2003) Proc. Natl. Acad. Sci. USA 100: 183-188; Miyoshi et al. (1998) J. Virol. 72: 8150-8157; Tisconia et al. (2003) Proc. Natl. Acad. Sci. USA 100: 1844-1848; and Pfeifer et al. (2002) Proc. Natl. Acad. Sci. USA 99: 2140-2145. Lentiviral vector kits are available from Invitrogen (Carlsbad, Calif.), based upon patents licensed from Cell Genesys, Inc.

VI. Selectable Marker Genes

It is frequently desirable to have a method for identifying cells that have successfully incorporated a nucleic acid construct of the present invention. This is preferably accomplished through the inclusion of a selectable marker gene into the vector used in the transformation process. An example of such a selectable marker is the puro^{r} gene depicted in FIG. 2. Selectable markers allow a transformed cell, tissue or animal to be identified and isolated by selecting or screening the engineered material for traits encoded by the marker genes present on the transforming DNA. For instance, selection may be performed by growing the engineered cells on media containing inhibitory amounts of the antibiotic to which the transforming marker gene construct confers resistance. Further, transformed cells may also be identified by screening for the activities of any visible marker genes (e.g., the β-glucuronidase, green fluorescent protein, luciferase, B or C1 genes) that may be present on the recombinant nucleic acid constructs of the present invention. Such selection and screening methodologies are well known to those skilled in the art.

Physical and biochemical methods may also be used to identify a cell transformant containing the gene constructs of the present invention. These methods include but are not limited to: 1) Southern analysis or PCR amplification for detecting and determining the structure of the recombinant DNA insert; 2) Northern blot, S-1 RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis, western blot techniques, immunoprecipitation, or enzyme-linked immunoassays, where the gene construct products are proteins; 5) biochemical measurements of compounds produced as a consequence of the expression of the introduced gene constructs. Additional techniques, such as in situ hybridization, fluorescence activated cell sorting (FACS), enzyme staining, and immunostaining, also may be used to detect the presence or expression of the recombinant construct in specific cells, organs and tissues. The methods for doing all these assays are well known to those skilled in the arts.

A number of additional selection systems may also be used, including but not limited to the herpes simplex virus thymidine kinase (Wigler, et al., Cell, 11:223 (1977)), hypoxanthine-guanine phosphoribosyltransferase (Szybalska & Szybalski, Proc. Natl. Acad. Sci. USA, 48:2026 (1962)), and adenine phosphoribosyltransferase (Lowy et al., Cell, 22:817 (1980)) genes can be employed in tk⁻, hgprt⁻ or aprt⁻ cells, respectively. Also, antimetabolite resistance can be used as the basis of selection for dhfr, which confers resistance to methotrexate (Wigler et al., Natl. Acad. Sci. USA, 77:3567 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418 (Colberre-Garapin et al., J. Mol. Biol., 150:1 (1981)); and hygro, which confers resistance to hygromycin (Santerre, et al., Gene, 30:147 (1984)). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047 (1988)); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.).

VII. Host Cells

The expression cassettes of the present invention can be used to transform any eukaryotic or prokaryotic cell for a variety of purposes including, but not limited to, amplification of the expression cassette sequence, reverse genomic studies and gene therapy. Preferred cell types include bone marrow stem cells and hematopoietic cells. These cell types are relatively easily removed and replaced from humans, and provide a self-regenerating population of cells for the propagation of the transferred expression cassette and studies on the effects of the encoded dsRNA on cellular metabolism. Such cells can be transfected/transduced in vitro or in vivo with retrovirus-based vectors encoding an expression cassette. Eukaryotic cell types that can serve as targets for vectors containing expression cassettes of the present invention include primary cell cultures, cell lines, yeast, and cellular populations in whole organs and organisms.

The invention is not limited to the type of organism or type of cell in which dsRNA is expressed. Any organism in which the function of a DNA sequence is sought to be determined is contemplated to be within the scope of the invention. Such organisms include, but are not restricted to, animals (e.g., vertebrates, invertebrates.), plants (e.g., monocotyledon, dicotyledon, vascular, non-vascular, seedless, seed plants), protists (e.g., algae, citliates, diatoms), and fungi (including multicellular forms and the single-celled yeasts).

In addition, any type of cell into which an expression vector may be introduced is expressly included within the scope of this invention. Such cells are exemplified by embryonic cells (e.g., oocytes, sperm cells, embryonic stem cells, 2-cell embryos, protocorm-like body cells, callous cells), adult cells (e.g., brain cells, fruit cells), undifferentiated cells (e.g., fetal cells, tumor cells), differentiated cells (e.g., skin cells, liver cells), dividing cells, senescing cells, cultured cells, and the like.

Host cells can be transformed with the disclosed vectors using any suitable means and cultured in conventional nutrient media modified as is appropriate for inducing promoters, selecting transformants, or detecting expression. Suitable culture conditions for host cells, such as temperature and pH, are well known. The concentration of plasmid used for cellular transfection is preferably titrated to limit the number of vectors encoding different affector siRNA molecules introduced into an individual cell.

Preferred eukaryotic host cells for use in the disclosed method include, but are not limited to, monkey kidney CVI line transformed by SV40 (COS-7, ATCC CRL 1651); human embryonic kidney line (293, Graham et al., J. Gen Virol., 36:59 (1977)); baby hamster kidney cells (BHK, ATCC CCL 10); Chinese hamster ovary-cells-DHFR (CHO, Urlaub and Chasin, Proc. Natl. Acad. Sci. (USA), 77:4216 (1980)); mouse sertoli cells (TM4, Mather, Biol. Reprod., 23:243-251 (1980)); monkey kidney cells (CVI ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL51); TRI cells (Mather et al., Annals N.Y. Acad. Sci, 383:44-68 (1982)); human B cells (Daudi, ATCC CCL 213); human T cells (MOLT-4, ATCC CRL 1582); and human macrophage cells (U-937, ATCC CRL 1593). The cells can be maintained according to standard methods well known to those of skill in the art (see, e.g., Freshney, Culture of Animal Cells, A Manual of Basic Technique, (3d ed.) Wiley-Liss, N.Y. (1994); Kuchler et al., Biochemical Methods in Cell Culture and Virology (1977), Kuchler, R. J., Dowden, Hutchinson and Ross, Inc. and the references cited therein). Cultured cell systems often will be in the form of monolayers of cells, although cell suspensions are also used.

In a preferred embodiment, one or more reporter genes are used to identify those cells that are successfully transfected or transduced. The same or a different reporter gene can be expressed by the expression cassette expressing the dsRNA to provide an indication of actual dsRNA expression.

VIII. Transfection Techniques

Within certain aspects of the invention, expression cassettes may be introduced into a host cell utilizing a vehicle, or by various physical methods. Representative examples of such methods include transformation using calcium phosphate precipitation (Dubensky et al., PNAS, 81:7529-7533 (1984)), direct microinjection of such nucleic acid molecules into intact target cells (Acsadi et al., Nature, 352:815-818 (1991)), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS, 89:6094 (1990)), lipofection (Felgner et al., Proc. Natl. Acad. Sci. USA, 84:7413-7417 (1989)), microprojectile bombardment (Williams et al., PNAS, 88:2726-2730 (1991)), polycation compounds such as polylysine, receptor specific ligands, liposomes entrapping the nucleic acid molecules, and spheroplast fusion whereby E. coli containing the nucleic acid molecules are stripped of their outer cell walls and fused to animal cells using polyethylene glycol.

Direct cellular uptake of oligonucleotides (whether they are composed of DNA or RNA or both) per se is presently considered a less preferred method of delivery because, in the case of siRNA and antisense molecules, direct administration of oligonucleotides carries with it the concomitant problem of attack and digestion by cellular nucleases, such as the RNases. The preferred mode for administration of the expression cassettes of the present invention takes advantage of known vectors (as discussed above) to facilitate the delivery of the expression cassette such that it will be expressed by the desired target cells.

Where the host cell is a plant cell, expression vectors may be introduced by particle mediated gene transfer (U.S. Pat. No. 5,584,807). Alternatively, an expression cassette may be inserted into the genome of plant cells by infecting plant cells with a bacterium, including but not limited to an Agrobacterium strain previously transformed with the expression vector which contains an expression cassette of the present invention (U.S. Pat. No. 4,940,838).

IX. siRNA Gene Libraries

One of the main applications of the present invention is the construction of a library of expression cassettes which may be used for expressing randomized dsRNAs and/or randomized siRNAs for purposes of Inverse Genomics® analysis. Such a library provides a highly efficient method for identifying unknown cellular genes whose silencing by an siRNA produces a detectable change in a phenotypic character of the cell system in which the siRNA gene library is expressed.

In general terms, this method involves transfecting or transducing a population of cells with a randomized siRNA expression library. One or more biological activities of the population of cells is then monitored. Cells showing a change in the monitored activity are isolated, and the expression cassettes containing the operative siRNA of interest selected. The siRNA of these cassettes can be expanded for subsequent rounds of screening. The sequence of the selected siRNAs from the first and/or subsequent rounds of screening is determined, and this data is then used for searching nucleic acid databases and/or for generating probes to probe for the target nucleic acid(s) associated with the alteration of the monitored character, or for use in other applications.

Construction of an siRNA gene library in accordance with the present invention requires the synthesis of nucleic acid sequences coding for siRNAs as described supra. The nucleic acid sequences can be known or random. When the sequence is random, a family of randomized sequences can be obtained comprising (theoretically) all base permutations possible for the randomized sequence length, from a single batch synthesis. In general, this means that ₄N different library members will be produced, where N=the number of nucleotides in each of the randomized sequences. The members of the library can then be cloned into a bacterial vector for amplification, or can be PCR amplified using techniques well known in the art. Sambrook et al., Molecular Cloning—A Laboratory Manual (2nd ed.) Vols. 1-3, Cold Spring Harbor Laboratory, Cold Spring Harbor Press, N.Y., (Sambrook) (1989); and F. M. Ausubel et al., (eds.) Current Protocols in Molecular Biology, Current Protocols, a joint venture between Greene Publishing Associates, Inc. (1994) and John Wiley & Sons, Inc. (1994 Supplement) (Ausubel).

Each randomized nucleic acid sequence is then ligated into an expression cassette of the invention such that one of the promoters will transcribe one strand of the randomized nucleic acid sequence, while the other promoter will transcribe the complementary strand after it has been synthesized, as described supra. The promoters preferably are modified pol III type III promoters, as described herein, having at least four bases of the promoter positioned 3′ to the TATA box substituted with adenylyl residues, and a second optional substitution of from 1 to 20 bases 5′ to the adenylyl residues and 3′ to the TATA box. The optional 1 to 20 base substitution can comprise a restriction site(s) or an operator sequence.

Once the nucleic acid sequence is positioned in the expression cassette or expression vector, its complementary strand is synthesized. This can be done enzymatically using the Klenow fragment of E. coli DNA polymerase I, or alternatively, the expression cassette can be incorporated into a vector that is then used to transform a competent cell line, with the missing complementary sequence being incorporated into the expression cassette by the cells' repair enzymes.

Alternative methods of forming the dsRNA expression library of the present invention involve synthesizing the complementary strand to the nucleic acid sequence prior to ligation of the nucleic acid sequence into the expression cassette. The resulting double-stranded molecule can then be ligated between the promoters of the expression cassette, for example, by blunt-end ligation.

In some embodiments of the invention, the 5′ end of the sequence is capped with a guanylyl residue or an adenylyl residue and the 3′ end with a cytosyl or thymidyl residue, respectively, the resulting guanylyl or adenylyl residues of each strand being the first transcribed base for the respective promoters of the dual promoter sequence.

In other embodiments of the invention, siRNA gene libraries of known sequence are produced. To produce such siRNA libraries, methods analogous to those described above are employed, with the nucleic acid sequences encoding the known siRNAs replacing the dsRNA coding sequence in the cassettes.

The expression cassettes of the library can be incorporated into a suitable vector either prior to, or after, insertion of the nucleic acid sequence. Suitable vectors for the library have been described supra.

Verification of siRNA Libraries

The siRNA gene libraries of the present invention may be verified both qualitatively and quantitatively. Qualitative verification involves transcribing in vitro the entire expression library in one reaction and then evaluating its ability to inhibit expression of a variety of different known genes, of both cellular and viral origin. In addition, the expression library can be subjected to DNA sequencing and a properly prepared library will result in equal band intensity across all four sequencing lanes for each randomized position.

Quantitative analysis involves statistical analyses of individual dsRNAs (picked from the expanded library and sequenced) to build confidence intervals for each base position in each molecule, thus allowing an evaluation of the complexity of the library without having to manually sequence each individual dsRNA coding sequence. The formula for a two-sided approximate binomial confidence interval is E=1.96 * square root(P * (1-P)/N), where P is the expected proportion of each nucleotide in a given position (which for DNA bases equals 25% or P=0.25), E is the desired confidence interval around P (i.e. P±E) and N is the required sample size (Callahan Associates Inc., La Jolla, Calif.). For example, if we need to know the proportion of each base within 5% (E=0.05), then the required sample size is 289.

Detecting Change in One or More Phenotypic Characteristics

As explained, an siRNA gene library may be introduced into a cell system of interest and the cell system monitored to detect a difference or change in one or more detectable phenotypic characteristics. The particular character (activity) and the method of measuring it vary with the kind of gene under examination. For example, the methods of the invention can be used to detect genes that mediate sensitivity and resistance to a selected defined chemical substance; examples include: drug toxicity genes; genes that encode resistance or sensitivity to carcinogenic chemicals; and genes that encode resistance or sensitivity to infections with specific viral and bacterial pathogens. The methods of the invention are also used to detect unknown genes that mediate binding to a ligand, such as hormone receptors, viral receptors, and cell surface markers. The methods of the invention are also used to detect unknown tumor suppressor, transformation, and differentiation genes.

Phenotypic changes can be morphologic, biochemical, or behavioral. Morphological changes typically are manifest in alterations in gross anatomy of the transfected organism. Biochemical changes may be determined by, for example, changes in the activity of known enzymes, rate of accumulation or utilization of certain substrates, protein patterns on two-dimensional polyacrylamide gel electrophoresis, etc. Such changes in response to siRNA expression suggest that the gene whose transcript is the target of the siRNA acts in the same pathway as the enzyme(s) whose activity is altered, or in a related pathway which either supplies substrate to these pathways, or utilizes products generated by them.

Molecular biological changes can be determined by, for example, differential display reverse transcription-PCR (DDRT-PCR). Such changes suggest that the gene whose expression is inhibited by the siRNA encodes a transcriptional regulatory molecule such as a transcription factor.

The DDRT-PCR method is based on the polymerase chain reaction, which is described by Mullis, et al., in U.S. Pat. Nos. 4,683,195, 4,683,202 and 4,965,188. Briefly, the PCR process consists of introducing a molar excess of two oligonucleotide primers to the DNA mixture containing the desired target sequence. The two primers are complementary to the respective strands of the double-stranded sequence. The mixture is denatured and then allowed to hybridize. Following hybridization, the primers are extended with a thermostable DNA polymerase so as to form complementary strands. The steps of denaturation, hybridization, and polymerase extension can be repeated as often as needed to obtain a relatively high concentration of a segment of the desired target sequence.

When DDRT-PCR is used, the target is mRNA; the mRNA is, however, treated with reverse transcriptase in the presence of oligo(dT) primers to make cDNA prior to the PCR process. The PCR is carried out with random primers in combination with the oligo(dT) primer used for cDNA synthesis. In theory, since only mRNA is (indirectly) amplified, only the expressed genes are amplified. Where two samples are to be compared, the amplified products are placed in side-by-side lanes of a gel; following electrophoresis, the products can be compared or “differentially displayed.”

Improved DDRT-PCR methods have been described in the art, including for example, the improvements described by E. Haag et al., “Effects of Primer Choice and Source of Taq DNA Polymerase on the Banding Patterns of Differential Display RT-PCR,” Biotechniques, 17:226-228 (1994). Another example is O. C. Ikonomov et al., “Differential Display Protocol With Selected Primers That Preferentially Isolate mRNAs of Moderate to Low Abundance in a Microscopic System,” Biotechniques, 20:1030-1042 (1996).

Yet another alternative is the determination of behavioral changes in an organism. Where the organism is unicellular, e.g., yeast, such changes may include light tropism, chemical tropism and the like, and would suggest that the gene whose expression is reduced by the presence of siRNA regulates these events. Where behavioral changes are observed in a multicellular organism, e.g., loss of spatial memory, aggressiveness, etc., such changes indicate that the gene whose transcript is targeted by the siRNA functions in a neural pathway involved in controlling such behavior.

As indicated above, the particular phenotypic characteristic under investigation determines the type of assay utilized. For example, the effects of siRNAs on nucleic acids that encode receptors (e.g., hormone or drug receptors, such as platelet-derived growth factor receptor is measured in terms of differences of binding properties, differentiation, or growth. Effects on transcription regulatory factors are measured in terms of the effect of siRNAs on transcription levels of affected genes. Effects on kinases are measured as changes in levels and patterns of phosphorylation. Effects on tumor suppressors and oncogenes are measured as alterations in transformation, tumorigenicity, morphology, invasiveness, adhesiveness and/or growth patterns. The list of types of gene function and phenotypes that are subject to alteration goes on: viral susceptibility—HIV infection; autoimmunity—inactivation of lymphocytes; drug sensitivity—drug toxicity and efficacy; graft rejection—MHC antigen presentation, etc. The monitoring of biological characteristics in gene function studies using the methods of the present invention is illustrated in Example 4.

Effects of siRNAs on cellular differentiation can be assayed by changes in cell growth/proliferation, changes in surface proteins (sort by FACS), loss or gain of adherence/differential trypsinization, changes in cell size (sort by FACS), etc. Thus, for example, PC12 cells whose differentiation is inhibited by siRNAs do not become post-mitotic and stop dividing.

Cell death is also a useful indicator. For example, cells that are drug resistant (e.g. multidrug resistant cancer cells) can be transfected or transduced with an siRNA expression library and assayed for cell death in the presence of a cytotoxic drug (e.g. a cancer therapeutic such as cisplatin, vincristine, methotrexate, doxorubicin, etc.).

The foregoing list of characters that may be monitored is illustrative and not intended to be exhaustivesince the variety of characters that can be screened in target acquisition studies is virtually limitless.

Use of Controls in Gene Identification Assays

It will be appreciated that where transfection or transduction with members of an siRNA expression library results in the alteration of a particular character/biological activity, the change is typically measured with reference to an “unchanged” negative control and, optionally, a deliberately changed “positive” control. The use of such controls is well known to those of skill in the art. Typically, negative controls are provided by an essentially identical cell, tissue, organ, or animal model that has not been transfected or transduced with the siRNA expression library. A measurable difference, preferably a statistically significant difference between the control and the assay system indicates that an siRNA has an effect.

It will be appreciated, however, that in selection systems, selection is its own control. Thus, for example, where tumorigenic cells live and normal cells die (e.g. on soft agar) or drug resistant cells live while drug sensitive cells die, the simple fact of survival can indicate a significant alteration in a phenotypic character.

Isolation of Cells Showing a Phenotypic Change and Recovery of the siRNA Gene

Cells showing a change in the monitored activity due to transfection/transduction with an siRNA may be isolated according to standard methods known to those of skill in the art. Cells in in vitro culture can simply be physically isolated and amplified, e.g. simply by spotting the appropriate transformed cells out into new culture medium, or they can be isolated visually where there is a visually detectable marker, or they can be mechanically isolated, e.g. by cell sorting (FACS). Where the cells are present in a tissue, organ, or organism, the cells can be isolated by any of these means after sacrifice of the organism, if necessary, and homogenization of the tissue or organs to obtain free cells in suspension.

The siRNA gene library can be recovered according to standard methods well known to those of skill in the art. Methods for recovery of plasmids (or other constructs) from bacterial hosts are described in. Sambrook et al., (1989) supra. and Ausubel et al., (ed.) (1987) supra.

After isolation and selection of the cells displaying the desired phenotype, it is possible to “rescue” the responsible siRNA expression cassettes (or portions thereof) from the selected cells. The rescued siRNA expression cassettes are used both for re-application to fresh cells to verify the siRNA-dependent phenotype and for direct sequencing of the siRNA expression cassette so as to identify the target gene.

In one approach, siRNA genes may be rescued from tissue culture cells by either PCR of genomic DNA or by rescue of the viral genome (e.g., either AAV or retrovirus). To rescue by PCR, cells are lysed in a lysis buffer containing a protease (e.g., proteinase K). The protease is then inactivated (e.g., by incubation at 95° C. for 5 minutes). The siRNA genes can then be isolated by PCR. Choice of PCR primers depends on the starting library vector and can be designed to amplify up to 1000 bp containing the siRNA sequence. The amplified siRNA gene fragment is then gel purified (agarose or PAGE).

This PCR product can be used for direct sequencing (fmole Sequencing Kit, Promega) or digested with appropriate restriction enzymes and re-cloned into a cloning or expression vector of the invention. This PCR rescue operation can be used to isolate not only single siRNA genes from a clonal cell population, but it can also be used to rescue a pool of siRNA genes present in a phenotypically-selected cell population. After the siRNA genes are re-cloned, the resulting plasmids can be used directly for target cell transfection or for production of a viral vector.

An alternative method for siRNA gene rescue involves “rescue” of the viral genome from the selected cells by providing all necessary viral helper functions. In the case of retroviral vectors, selected cells are transiently transfected with plasmids expressing the retroviral gag, pol and amphotropic (or VSV-G) envelope proteins. Over the course of several days, the stably expressed LTR transcript containing the siRNA gene is packaged into new retroviral particles, which are then released into the culture supernatant. It is also possible to “rescue” the viral genome by infecting the transduced cells with wild-type, replication-competent retrovirus. In the case of AAV, selected cells are transfected with a plasmid expressing the AAV rep and cap proteins and co-infected with wild type adenovirus. Here the stably-integrated AAV genome is excised and re-packaged into new AAV particles. At the time of harvest, cells are lysed by three freeze/thaw cycles and the wild type adenovirus in the crude lysate is heat inactivated at 55° C. for 2 hours. The resulting virus-containing media (from either the retroviral or AAV rescue) is then used to directly transduce fresh target cells to both verify phenotype transfer and to subject them to additional rounds of phenotypic selection if necessary to enrich further for the phenotypic siRNA genes. Similar to the PCR method described above, viral rescue of siRNA genes allows for rescue of either a single siRNA gene or “pools” of siRNA genes from non-clonal populations.

As indicated above, the rescued siRNA genes are used both for re-application to fresh cells to verify siRNA-dependent phenotype and for direct sequencing of the siRNA genes to enable identification of the target gene(s) associated with the phenotypic change. In addition, the rescue of “pools” of siRNA genes from non-clonal populations provides an enriched siRNA expression library that can be used for subsequent rounds of selection.

Identification of Genes Silenced by siRNA

Once the siRNA genes have been isolated, they can be sequenced and their sequences used to search sequence databases for the nucleic acid targeted by the siRNA. A number of algorithms suitable for comparing nucleotide sequence similarity are available to those in the art. For example, preferred algorithms include the BLAST and BLAST 2.0 algorithms, which are described in Altschul et al., Nuc. Acids Res., 25:3389-3402 (1977) and Altschul et al., J. Mol. Biol., 215:403-410 (1990), respectively. Software for performing BLAST analyses is publicly available through the National Center for Biotechnology Information (at its website ncbi.nlm.nih.gov). An alternative to the BLAST program is the GCG (Genetics Computer Group, Program Manual for the GCG Package, Version 7, Madison, Wis.) PILEUP program. PILEUP creates a multiple sequence alignment from a group of related sequences using progressive, pair wise alignments to show relationship and percent sequence identity. It also plots a tree or dendrogram showing the clustering relationships used to create the alignment. PILEUP uses a simplification of the progressive alignment method of Feng and Doolittle, J. Mol. Evol., 35:351-360 (1987).

Should a database search fail to identify the siRNA target, the siRNA sequence can be used to construct probes and primers for identifying and isolating target mRNAs and genes. For example, the siRNA sequences can be used to construct radiolabelled probes for detecting mRNAs, cDNAs and genomic sequences of target molecules. Samples of endogenous nucleic acids can, for example, be partially purified by a variety of methods known in the art, and the fraction containing the target nucleic acid identified as that fraction capable of hybridizing to a probe having the siRNA sequence.

An exemplary method for isolating target nucleic acids of siRNAs can be achieved using the siRNA nucleotide sequence to construct primers that are then used in polymerase chain reaction, or other in vitro amplification methods. (see U.S. Pat. Nos. 4,683,195 and 4,683,202; PCR Protocols: A Guide to Methods and Applications (Innis et al., eds, 1990)). Nucleotides amplified by the PCR reaction can be purified from agarose gels and cloned into an appropriate vector.

Particularly useful PCR techniques include 5′ and/or 3′ RACE techniques, both being capable of generating a full-length cDNA sequence from a suitable cDNA library (Frohman, et al., Proc. Natl. Acad. Sci. USA, 85:8998-9002 (1988)). The strategy involves using specific oligonucleotide primers, based on the siRNA sequence, for PCR amplification of the target nucleotide. Kits for performing PCR amplification, including 3′ and 5′ RACE techniques, using sequence specific primers are commercially available (PanVera, Discovery Center, Madison, Wis., 3′ and 5° Full RACE Core Sets, Prod #s TAK 6121 and 6122; Invitrogen Corporation, Carlsbad, Calif., CAT. NO. 18373019, CAT. NO. 10630010).

X. Therapeutic Uses for the Invention

In addition to the uses noted above, the expression cassettes and vector constructs of the present invention may be used as therapeutics, research reagents, and for gene therapy applications.

For therapeutic use, an animal suspected of having a genetically-based disease is treated by administering expression cassettes producing siRNA in accordance with this invention. Persons of ordinary skill can easily determine optimum dosages, dosing methodologies and repetition rates. Such treatment is generally continued until either a cure or a diminution in the diseased state is achieved. Long term treatment is likely for some diseases. Treatment of viral diseases, including HIV, are particularly preferred therapeutic applications of the expression cassettes of the present invention.

Organismal cellular transduction provides methods for combating chronic infectious diseases such as AIDS, caused by HIV infection, as well as non-infectious diseases such as cancers. Yu et al., Gene Therapy, 1:13-26 (1994) and the references therein provides a general guide to gene therapy strategies for HIV infection. See also, Sodroski et al., PCT/US91/04335. Wong-Staal et al., WO/94/26877, describe retroviral gene therapy vectors.

Suitable vectors containing expression cassettes producing siRNA according to the present invention, and in some applications naked siRNAs produced according to the present invention, can be used directly in combination with a pharmaceutically acceptable carrier to form a pharmaceutical composition suited for treating a patient.

Direct delivery involves the insertion of the expression cassettes or naked siRNAs into the target cells, usually with the help of lipid complexes (liposomes) to facilitate the crossing of the cell membrane and other molecules, such as antibodies or other small ligands, to maximize targeting. Because of the sensitivity of RNA to degradation, in many instances, directly delivered siRNA molecules may be chemically modified, making them nuclease-resistant, as described above. This delivery methodology allows a more precise monitoring of the therapeutic dose.

Vector-mediated delivery involves the infection of the target cells with a self-replicating or a non-replicating system, such as a modified viral vector or a plasmid, which produces a large amount of the siRNA encoded in a sequence carried in the expression cassette of the vector as described herein. Targeting of the cells and the mechanism of entry may be provided by the virus, or, if a plasmid is being used, methods similar to the ones described for direct delivery of siRNA molecules can be used. Vector-mediated delivery produces a sustained amount of siRNA. It is substantially cheaper and requires less frequent administration than a direct delivery such as intravenous injection of the siRNA molecules.

The direct delivery method can be used during the acute critical stages of infection. Preferably, intravenous or subcutaneous injection is used to deliver siRNA molecules directly. It is essential that an effective amount of oligonucleotides be delivered in a form that minimizes degradation of the oligonucleotide before it reaches the intended target site.

Most preferably, the pharmaceutical carrier specifically delivers the siRNA to affected cells. For example, hepatitis B virus affects liver cells, and therefore, a preferred pharmaceutical carrier delivers anti-hepatitis siRNA molecules to liver cells.

Expression cassettes producing siRNAs of the invention are useful as components of gene therapy vectors. For example, retroviral vectors packaged into HIV envelopes primarily infect CD4⁺ cells, (i.e., by interaction between the HIV envelope glycoprotein and the CD4 “receptor”) including, non-dividing CD4⁺ cells such as macrophage.

XI. Kits

In still another embodiment, this invention provides kits for the practice of the methods of this invention. The kits preferably comprise one or more containers containing an siRNA gene library and/or siRNA gene vector library of this invention. The kit can optionally include buffers, culture media, vectors, sequencing reagents, labels, antibiotics for selecting markers, and the like.

The kits may additionally include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of non-critical parameters that could be changed or modified to yield essentially similar results.

### EXAMPLES

### Example 1

### Construction of a Randomized siRNA Gene Vector Library

This example illustrates methods for constructing a randomized siRNA gene vector library, wherein expression of the library is under the control of two opposing U6 snRNA promoters.

The first step in constructing the randomized siRNA gene vector library is to create two mutated U6 snRNA promoter fragments using either human genomic DNA or a cloned wild type U6 promoter DNA as the template for PCR amplification. To create the first mutated U6 promoter, a PCR fragment is generated using an upstream primer modified to contain a Hind III site outside of the 5′ end of the U6 promoter (upstream of -265) and a downstream primer modified to contain Not I and Xho I restriction sites at the 3′ end of the U6 promoter. These modifications create the mutations in the promoter downstream of the “TATA box”.

| | | |
|---|---|---|
| Hind III U6-265: | 5′-TGCTAAGCTTAAGGTCGGGCAGGAAGAG-3′ | (SEQ ID NO:1) |
| NX U6-20: | 5′-ATGCTCGAGCGGCCGCAGATATATAAAGCCAA-3′ | (SEQ ID NO:2) |

The second mutated U6 promoter PCR fragment is generated using an upstream primer modified to contain an Mlu I site outside of the 5′ end of the promoter (upstream of -265) and a downstream primer modified to contain Sph I and Xho I restriction sites at 3′ end of the U6 promoter (downstream of the TATA box).

| | | |
|---|---|---|
| Mlu I U6-265: | 5′-TGCTACGCGTAAGGTCGGGCAGGAAGAG-3′ | (SEQ ID NO:3) |
| SX-U6-20: | 5′-ATGCTCGAGCATGCAGATATATAAAGCCAA-3′. | (SEQ ID NO:4) |

Following amplification and purification, the first PCR fragment, comprising the first mutated U6 snRNA promoter, is digested with Xho I and Hind III. The second mutated promoter fragment is digested with Mlu I and Xho I. The two digested fragments are then ligated using T4 DNA ligase. The resulting ligation product, comprising the two mutated promoters facing each other, is inserted into a vector, pLPR-1kb (FIG. 2), from which the Hind III-Mlu I fragment is removed by Hind III and Mlu I digestion and gel isolation. The final product is the expression vector, pLPR-2U6, which contains Not I, Xho I and Sph I sites and is used to express the siRNA gene library as described below.

Optionally, a second expression vector, pLPR-2U6-stuffer, is created from pLRP-2U6 to improve the convenience of the subsequent cloning steps. To create the pLPR-2U6-stuffer, a non-relevant 2 kb stuffer sequence is inserted in the Xho I site of pLPR-2U6. This insertion permits ready detection and isolation of the digested vector sequence because the restriction digestion produces two distinct, well separated bands on an agarose gel. Both pLPR-2U6 and pLPR-2U6-stuffer plasmids may be used as expression vectors for cloning of the randomized siRNA genes.

After creating the vector, an siRNA gene library (siRNA-LIB) is synthesized, utilizing techniques known in the art. Each chemically synthesized oligo DNA has the basic structure:

| | |
|---|---|
| 5′-pGGCCGCGGACGAAAAAAAGnnnnnnnnnnnnnnnnnnnnCTTTTTGACGACGGCGCATG-3′ | (SEQ ID NO:5) |

Each oligo has the following features:

1) a phosphorylated 5′-end;

2) the sequence GGCC at the 5′ end, which functions in subsequent cloning steps by annealing to the Not I generated 3′ overhang of the cut pLRP-2U6 or pLRP-2U6-stuffer vector;

3) a sequence of seven nucleotides corresponding to the wild-type human U6 promoter;

4) a sequence of five As (AAAAA), which is the reverse complement of the pol III promoter type III termination signal (e.g. TTTTT), placed immediately upstream of the siRNA, replacing the last five nucleotides of the natural promoter;

5) an siRNA gene sequence with a basic structure conforming to the sequence GnnnnnnnnnnnnnnnnnC, where n is randomized, i.e. is any of one of the four nucleotides (dT, dA, dG, dC) at any position;

6) a sequence of five Ts (TTTTT), which comprises the pol III promoter type III termination signal, immediately downstream of the siRNA gene sequence, replacing the last few nucleotides (-1 to -5) of the second U6 promoter;

7) an arbitrary sequence of nine nucleotides which are not complementary to the corresponding region of the opposite promoter; and

8) the sequence CATG at the 3′ end, which functions in subsequent cloning steps to permit annealing of the oligo with the SphI generated 3′ overhang of the cut pLPR-2U6 vector or pLPR-2U6-stuffer vector.

Two additional universal oligos are also chemically synthesized, as follows:

Univ-1 (Not I): 5′-CTTTTTTTCGTCCGC-3′ (SEQ ID NO:6); and

Univ-2 (Sph I): 5′-pCGCCGTCGTCAAAAAG-3′ (SEQ ID NO: 7), where the 5′-end is phosphorylated.

The random siRNA gene library (siRNA-LIB) is then inserted into the cloning vector (pLPR-2U6-stuffer) by annealing to Univ-1 and Univ-2 and ligating the annealed oligos to the vector from which the Not I/Sph I stuffer fragment has been removed. The molar ratio for the oligos and vector DNAs are: Univ-1:Univ-2:siRNA-LIB:pLPR=100:100:5:1. The ligated products are then transformed into electro-competent bacteria (DH12S Invitrogen, Carlsbad, Calif., USA), with the transformation conditions optimized as is known in the art to maximize the complexity of the library. Single strand gaps in the ligated product are filled-in by the bacteria in vivo. Alternatively, the single strand gaps in the ligated product may be filled-in in vitro using Klenow DNA polymerase (Promega, Madison, Wis., USA) and four dNTPs. The transformed bacteria are then plated on LB agar plates at a density of less than 1×10⁵ per 150 mm plate and cultured overnight. The overnight-cultured cells are then harvested and used as library bacterial stock. Optimally, more than 5×10⁷ total clones are generated.

### Example 2

### Expression of a Specific siRNA for Down-Regulation of Gene Expression

This example demonstrates the use of the vector of Example 1 to express a specific siRNA which results in down-regulation of gene expression. Specifically, this example illustrates down-regulation of firefly luciferase in a breast cancer cell line.

A vector is constructed as described in Example 1. After creating the vector, the following oligonucleotides, which have the same basic structure as the oligos comprising the siRNA gene library of Example 1, are chemically synthesized:

| | | |
|---|---|---|
| siRNA-lucB: | 5′-pGGCCGCGGACGAAAAAAAGTGCGCTGCTGGTGCCAACCCTTTTTGACGACGGCGCATG-3′ | (SEQ ID NO:8) |
| siRNA-Scramble: | 5′-pGGCCGCGGACGAAAAAAAGCGCGCTTTGTAGGATTCGCCTTTTTGACGACGGCGCATG-3′ | (SEQ ID NO:9) |

The first of these oligos serves as the template for the creation of a luciferase specific siRNA gene, and the second provides a control siRNA gene. As described in Example 1, each of these oligos is annealed with the two universal oligos: Univ-1 and Univ-2, and ligated to the pLPR-2U6-stuffer vector from which the NotI/SphI stuffer fragment is removed. Resulting single strand gaps are then filled in by bacteria after transformation

The resulting plasmids, pLPR-2U6-lucB-siRNA and pLPR-2U6-scramble-siRNA, are each separately introduced into the MCF7-Luc cell line by transfection. This cell line is a breast cancer cell line that expresses firefly luciferase. Two days after transfection, both cell lysates and total RNA are prepared, from each of the transfected cell lines. The level of luciferase activity is measured using a luciferase assay kit (Promega, Madison, Wis., USA), and total RNA is analyzed by Taqman® (Li, Q. et al., Nucleic Acids Research, 28:2605 (2000)). Alternatively, 10 days after transfection, stable transfectants are selected by puromycin selection (1 ug/ul) and the luciferase activity and total mRNA levels are measured as before. The luciferase assay shows down-regulation of luciferase activity in the cell line transfected with pLPR-2U6-lucB-siRNA as compared with the control, and this is confirmed by a reduction in mRNA level, as shown by the Taqman® assay.

### Example 3

### Generating an Inducible System for Expression of a Randomized siRNA Library or a Specific siRNA Gene

This example illustrates the generation of an inducible siRNA system for expression of either a randomized siRNA gene library or a specific siRNA gene. In this example, the regulatory sequences from the tetracycline operon of E. coli Tn10 are used to control expression of a human U6 snRNA promoter driven siRNA gene or siRNA gene library.

To generate the inducible promoter, the constructs in Examples 1 and 2 are further modified to express the siRNA gene only when tetracycline is present in the media. The steps involved in constructing the tetracycline regulated expression vector are almost identical to those of Example 1 and Example 2, except for two additional requirements. First, the tetracycline operator sequences are used to replace wild-type promoter sequences between the TATA box and the proximal sequence element (PSE) of the U6 promoter region. This is accomplished by incorporating the tetracycline operator sequences into the primer that is used to PCR amplify the U6 promoter sequences (see below). Second, in addition to the siRNA gene, a tetracycline repressor gene is provided in the host cells either in cis or in trans.

Thus, the expression vector for these experiments employs two mutated U6 promoters facing each other, and is constructed as described in Example 1, except that instead of using primers NX U6-20 and SX-U6-20 as in Example 1, this cloning vector is created using the following primers:

| | | |
|---|---|---|
| NX-U6-Tet-o: | 5′-TGCTCGAGCGGCCGCAGATATATAACTCTATCAATGATAGAGTACTTTCAAGTTACGGT-3′ | (SEQ ID NO:10) |
| SX-U6-Tet-o: | 5′-ATGCTCGAGCATGCAGATATATAACTCTATCAATGATAGAGTACTTTCAAGTTACGGT-3′ | (SEQ ID NO:11) |

The tetracycline operator sequences (indicted in italics) are incorporated into the primers such that the promoter resulting from the PCR will have a tetracycline operator inserted between the TATA box and the proximal sequence element (PSE) (see FIG. 3). The specific siRNA gene or the randomized gene library is then cloned into the tetracycline inducible expression vector as described in Example 1 and Example 2.

When the tetracycline repressor gene is provided in trans, in addition to the siRNA gene or gene library vector (e.g., pLPR-siRNA(luc)-tet), a separate vector expressing the repressor, such as pTET-ON (Clontech, Calif., USA) is introduced into the host at the same time. When the tetracycline repressor gene is provided in cis, the repressor gene is cloned into the pLPR vector under control of the pol III promoter in LTR and the final construct is: pLPR-siRNA(luc)-tet-rep.

After construction of the vector containing an inducible promoter (e.g., pLPR-siRNA(luc)-tet-rep), as described above, the cell system (e.g., MCF7-luc) is stably transfected and the stable transfectants are treated with tetracycline for 48 hours. Controls which are not treated with tetracycline are set up in parallel. The luciferase activity and luciferase mRNA are measured as described in Example 2.

It will be appreciated that in the absence of induction by tetracycline, siRNA expression is suppressed due to binding of the tetracycline operator sequence by the repressor. Therefore, an increase in luciferase activity is readily detected. However, when the cells are treated with tetracycline for 48 hours, siRNA gene expression is induced, and luciferase activity is reduced by comparison with untreated control cells.

### Example 4

### Using an siRNA Gene Library to Identify a Gene Associated with a Specific Phenotype

This example illustrates how an siRNA gene library is used to identify a gene involved in a specific phenotype in a cell system of interest. Specifically, in this example, a gene involved in the down-regulation of CD4 surface molecule gene expression is detected using fluorescence activated cell sorting (FACS) of cells transfected with an siRNA gene library.

The human T-cell line, Molts-4, expresses the CD4 molecule on its surface. CD4 is readily detected, and its quantity is measured using fluorescence labeled anti-CD4 antibody and FACS analysis. Cells with differing levels of surface CD4 expression can also be readily separated from each other by FACS sorting.

To identify an siRNA that down-regulates surface CD4 expression, the siRNA gene library from Example 1 or Example 3 is introduced into Molts-4 cells by transfection or retroviral transduction. The transfected/transduced cells are then FACS sorted according to fluorescence intensity, which is a reflection of surface CD4 expression. The low CD4-expressors in the transfected/transduced population are selected. The siRNA genes are rescued by PCR, re-cloned and re-introduced into Molts-4 cells. A few rounds of the same selection scheme are performed to enrich for the siRNAs that down-regulate CD4 expression.

The isolated siRNAs are those that directly target CD4 mRNA or alternatively, are mRNAs encoding proteins that otherwise regulate CD4 expression. Based on the sequence information of the siRNAs, the target gene information is determined by BLAST searching of public or private databases or by direct gene cloning using the identified siRNA sequences as probes.

### Example 5

### Down-Regulation of p53 Gene Expression using a Human U6/Murine U6 Dual Promoter Expression Cassette

This example shows the use of a human U6/murine U6 dual promoter retroviral expression vector for the expression of an siRNA that silences p53 gene expression. A vector was constructed as in Example 1, with the modifications described below, using pTZ U6+1 (Lee et al. (2002) Nat. Biotechnol. 20: 500-505) and pSilencer 1.0-U6 (Ambion, Austin, Tex.) as sources of the human and murine U6 promoters, respectively.

The primers used for PCR amplification were:

| | | |
|---|---|---|
| 5′ hU6 + BamHI: | 5′-TGCTGGATCCAAGCTTAAGGTCGGGCAGGAAGAG-3′ | (SEQ ID NO:12) |
| 3′ hU6 + FseI/XhoI: | 5′-GCATGCTCGAGGCCGGCCGATATATAAAGCCAAGAAATCG-3′ | (SEQ ID NO:13) |
| 5′ mU6 + BamHI/XbaI: | 5′-TCTAGAGAACTAGTGGATCCGACGCC-3′ | (SEQ ID NO:14) |
| 3′ mU6 + AscI/XhoI: | 5′-gccgctcgaggcgcgccATATTTATAGTCTCAAAACACAC-3′ | (SEQ ID NO:15) |

Both PCR products were ligated into the pCR-Blunt II-TOPO vector (Invitrogen, Carlsbad, Calif.) to generate pSD53 (human U6) and pSD96 (murine U6). A ˜110 bp XhoI/XbaI fragment from one of the pSD96 clones was then ligated into the ˜3.7 kb XhoI/XbaI fragment of a pSD53 clone in which the BamHI sites were 47 bp apart. The ˜560 bp BamHI/BamHI fragment of the resulting vector contained the human U6/murine U6 opposing promoter cassette.

The human U6/murine U6 opposing promoter cassette (BamHI/BamHI fragment) was inserted into a self-inactivating retroviral vector, pQCXIP (Clontech, Palo Alto, Calif.), modified to contain a unique BamHI site within the U3 region of the 3′ LTR. The MCS and IRES regions of this vector were also removed; however, expression of the puromycin resistance gene was still driven by the CMV promoter. A similar retroviral vector has been used to express hairpin siRNAs from a single pol III promoter (Barton and Medzhitov (2002) Proc. Natl. Acad. Sci. USA 99: 14943-14945).

Oligos encoding siRNAs against p53 and luciferase (control) were synthesized as follows:

p53 siRNA Oligo:

| | |
|---|---|
| 5′-pCCAGGACGACAAAAAgactccagtggtaatctacTTTTTAGGCTTTTCGG-3′ | (SEQ ID NO:16) |

Control (Luciferase) siRNA Oligo:

| | |
|---|---|
| 5′-pCCAGGACGACAAAAAgtgcgctgctggtgccaacccTTTTTAGGCTTTTCGG-3′ | (SEQ ID NO:17) |

These oligos have the same basic structure as the oligos comprising the siRNA gene library of Example 1 except that the GGCC sequence at the 5′ end and the CATG sequence at the 3′ end were replaced by CC and GG, respectively, reflecting the change from NotI/SphI cloning sites to FseI/AscI. The sequences of the universal oligos were also modified as follows:

| | |
|---|---|
| Univ-1(FseI): | |
| 5′-CTTTTTGTCGTCCTGGCCGG-3′ | (SEQ ID NO:18) |
| Univ-2(AscI): | |
| 5′-pCGCGCCGAAAAGCCTAAAAAG-3′ | (SEQ ID NO:19) |

Each of the siRNA-encoding oligos was annealed to the universal oligos and ligated into the FseI/AscI-digested opposing promoter cassettes in the retroviral vector as described in Example 1.

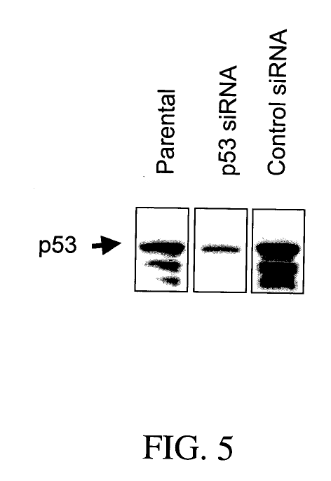

VSV-G pseudotyped retrovirus was packaged by co-transfecting a commercially available packaging cell line (Clontech, Palo Alto, Calif.) with the recombinant vector bearing the opposing promoter cassette and an expression vector for VSV-G protein. MCF-7 cells were then transduced with the retroviral vector. Following seven days of selection with puromycin, lysates were prepared and the p53 protein level was analyzed by western blot. As can be seen from FIG. 5, a significant knock-down of p53 gene expression was obtained.

### Example 6

### Comparison of Down-Regulation of p53 Gene Expression using a Human U6/Murine U6 Dual Promoter Expression Cassette and a Single U6 Murine Promoter Expression Cassette

This example compares the efficacy of down-regulation of p53 gene expression using two different types of expression cassettes to express p53 siRNA: a human U6/murine U6 dual promoter expression cassette in accordance with the invention, and a single murine U6 promoter cassette for expression of a hairpin siRNA. The same experimental procedures were followed as described above in connection with Example 5, except that A431 (rather than MCF-7) cells were transduced, and in addition to the retroviral vector, each expression cassette was also inserted into a self-inactivating lentiviral vector at a position between the HIV-1 DNA Flap element and an SV40 promoter-puromycin^{r} cassette. A similar lentiviral vector has been used to express hairpin siRNAs from a single pol III promoter (Qin et al. (2003) Proc. Natl. Acad. Sci. USA 100: 183-188).

For the single promoter cassette, the p53 siRNA was expressed from a single pol III promoter vector as described, e.g., in Brummelkamp et al. (2002) Science 296: 550-553; Paul et al. (2002) Nat. Biotechnol. 20: 505-508; Paddison et al. (2002) Genes and Development 16: 948-958; Yu et al. (2002) Proc. Natl. Acad. Sci. USA 99: 6047-6052).

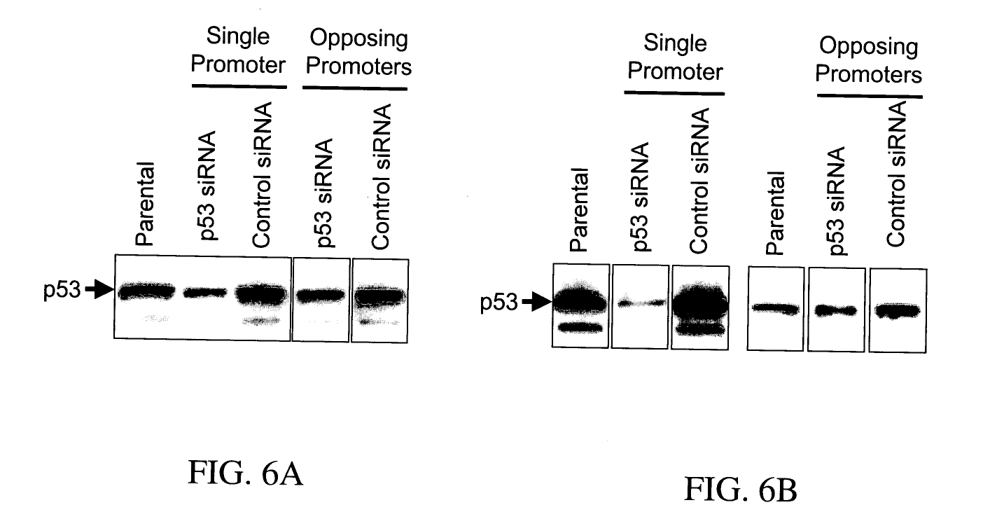

The results of these experiments are shown in FIGS. 6A and 6B. As can be seen, both p53 siRNA expression cassettes caused substantial specific silencing of p53 when delivered by either the retroviral or the lentiviral vector.

1. A DNA expression cassette comprising a double-stranded randomized DNA sequence between 16-25 bases long having a first and a second end, each end operably linked to a pol III promoter having a TATA box, wherein each of the promoters is modified by substitution of:
a. at least four consecutive adenylyl residues positioned 3′ to the TATA box; and,
b. from 0 to 20 bases 5′ to the at least four consecutive adenylyl residues and 3′ to the TATA box;
whereby transcription of the double stranded randomized DNA sequence from the promoters produces a dsRNA.

2. The DNA expression cassette of claim 1, wherein the 0 to 20 bases is at least one base and comprises a restriction site.

3. The DNA expression cassette of claim 1, wherein the promoters are the same.

4. The DNA expression cassette of claim 1, wherein the promoters are different.

5. The DNA expression cassette of claim 1, wherein the promoters are selected from the group consisting of H1 RNA promoters, U6 snRNA promoters, promoters for tRNA genes, and promoters for the adenovirus VA genes.

6. The DNA expression cassette of claim 1, wherein the randomized DNA sequence is between 17-23 bases long.

7. The DNA expression cassette of claim 1, wherein a first base transcribed in each strand of the randomized DNA sequence is G or A.

8. The DNA expression cassette of claim 1, further comprising an inducible operator sequence 5′ to the TATA box.

9. The DNA expression cassette of claim 8, wherein the inducible operator sequence is the tet O operator.

10. The DNA expression cassette of claim 1, further comprising a viral particle for packaging a nucleic acid comprising the expression cassette.

11. A self-replicating DNA comprising the DNA expression cassette of claim 1.

12. A library of DNA expression cassettes, each expression cassette comprising a double-stranded randomized DNA sequence between 16-25 bases long having a first and a second end, each end operably linked to a pol III promoter having a TATA box, wherein each promoter is modified by substitution of:
a. at least four consecutive adenylyl residues positioned 3′ to the TATA box; and,
b. from 0 to 20 bases 5′ to the at least four consecutive adenylyl residues and 3′ to the TATA box;
whereby transcription of the double stranded randomized DNA sequence from the promoters of each DNA expression cassette produces a different dsRNA.

13. The library of claim 12, wherein each of the randomized DNA sequences is between 17-23 bases long.

14. The library of claim 12, wherein the promoters are inducible.

15. The library of claim 12, wherein each DNA expression cassette is packaged in a viral particle.

16. The library of claim 12, wherein each DNA expression cassette is included in a cell genome.

17. The library of claim 12, wherein each DNA expression cassette is self-replicating.

18. A method for producing a library of DNA expression cassettes for expressing dsRNA having randomized sequences, the method comprising:
a. synthesizing a plurality of single-stranded randomized DNA sequences between 16 and 25 bases long, having a 5′ and a 3′ end;
b. constructing a plurality of expression vectors, each having a first and a second pol III promoter with a TATA box, wherein the first promoter is oriented to initiate transcription in the direction of the second promoter and the second promoter is oriented to initiate transcription in the direction of the first promoter, each promoter modified by substitution of:
i. at least four consecutive adenylyl residues positioned 3′ to the TATA box; and,
ii. from 0 to 20 bases 5′ to the at least four consecutive adenylyl residues and 3′ to the TATA box;
c. inserting one of the plurality of single-stranded randomized DNA sequences between the first promoter and the second promoter of each expression vector wherein the single-stranded randomized DNA sequence is operably linked to the first promoter; and
d. generating a DNA sequence complementary to each single-stranded randomized DNA sequence, the complementary DNA sequence being operably linked to the second promoter.

19. The method of claim 18, wherein the 0 to 20 bases of the constructing step is at least one base and comprises at least one restriction site.

20. The method of claim 18, wherein the generating step further comprises transforming competent bacteria with the plurality of expression vectors comprising the single stranded randomized DNA sequences.

21. The method of claim 18, wherein the generating step comprises in vitro synthesis of a DNA sequence complementary to each of the plurality of single-stranded randomized nucleic acid sequence using Klenow polymerase.

22. The method of claim 18, wherein the constructing step further comprises insertion of a guanylyl residue at the 5′ end of each single-stranded randomized DNA sequence and a cytosyl residue at the 3′ end, or an adenylyl residue at the 5′ end of each single-stranded randomized DNA sequence and a thymidyl residue at the 3′ end.

23. A method of correlating expression of a transcription sequence for an siRNA with a phenotypic change resulting from inhibiting expression of a cellular gene by the siRNA, where expression of the cellular gene is not previously characterized as contributing to the phenotypic change, the method comprising:
a. introducing to a cell population a library of exogenous randomized siRNAs, wherein each siRNA is produced from an expression cassette comprising a double-stranded randomized DNA sequence between 16-25 bases long, having a first end and second end, each end operably linked to a pol III promoter having a TATA box, wherein each promoter is modified by substitution of:
i. at least four consecutive adenylyl residues positioned 3′ to the TATA box; and,
ii. from 0 to 20 bases 5′ to the at least four consecutive adenylyl residues and 3′ to the TATA box;
b. detecting a phenotypic difference between the cells of the population introduced to the library of siRNAs and those cells not introduced to the library; and
c. identifying the siRNA of the library responsible for the phenotypic change.

24. The method of claim 23, further comprising isolating the siRNA of the library responsible for the phenotypic change.

25. The method of claim 23, wherein the introducing step comprises transducing the cell population by means of a viral transduction system.

26. The method of claim 23, wherein the detecting step comprises observation of a difference in cellular growth between the cells of the population introduced to the library of siRNAs and those cells not introduced to the library.

27. The method of claim 23, wherein the detecting step comprises co-expression of a detectable marker by the cells of the population introduced to the library of siRNAs.

28. The method of claim 27, wherein the detectable marker is selected from the group comprising a fluorescent protein, a cell surface protein, and a drug resistance gene.

29. The method of claim 23, wherein the cell population of the introducing step is a eukaryotic cell population.

30. The method of claim 23, wherein the phenotypic difference of the detecting step comprises inhibition of cell division, or viral gene expression, or excretion of an extracellular protein, or expression of a cell surface marker, or a genetic suppressor, or a signal transduction pathway, or cell death.
